(19) United States
(12) Patent Application Publication
(10) Pub. No.: US 2007/0161665 A1
(43) Pub. Date: Jul. 12, 2007

(54) Cancer treatment method

(76) Inventors: Inderjit Kumar Dev, Durham, NC (US); Tona Morgan Gilmer, Durham, NC (US); Cliford Nelson Rhodes III, Research Triangle Park, NC (US); Robert L. Tansik, Research Triangle Park, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/595,691

(22) PCT Filed: Nov. 5, 2004
(86) PCT No.: PCT/US04/37027

§ 371 Date: May 5, 2006

Related U.S. Application Data
(60) Provisional application No. 60/518,212, filed on Nov. 7, 2003.

(51) Int. Cl. A61K 31/519; A61K 31/517

(52) U.S. Cl. 514/264.11; 514/266.4; 514/266.24

(57) ABSTRACT

The present invention relates to a method of treating cancer in a mammal and to pharmaceutical combinations useful in such treatment. In particular, the method relates to a cancer treatment method that includes administering an erb family inhibitor and a PI3K and/or Akt inhibitor to a mammal suffering from cancer.

### BACKGROUND OF THE INVENTION

The present invention relates to a method of treating cancer in a mammal and to pharmaceutical combinations useful in such treatment. In particular, the method relates to a cancer treatment method that includes administering an erbB-2 and/or an EGFR inhibitor with a PI3K or Akt inhibitor to a mammal suffering from a cancer.

Effective chemotherapy for cancer treatment is a continuing goal in the oncology field. Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, 1993). In particular, cellular signalling from the growth factor receptors of the erbB family.

There is significant interaction among the ErbB family that regulates the cellular effects mediated by these receptors. Six different ligands that bind to EGFR include EGF, transforming growth factor, amphiregulin, heparin binding EGF, betacellulin and epiregulin (Alroy & Yarden, 1997; Burden & Yarden, 1997; Klapper et al., 1999). Heregulins, another class of ligands, bind directly to HER3 and/or HER4 (Holmes et al., 1992; Klapper et al., 1997; Peles et al., 1992). Binding of specific ligands induces homo- or heterodimerization of the receptors within members of the erbB family (Carraway & Cantley, 1994; Lemmon & Schlessinger, 1994). In contrast with the other ErbB receptor members, a soluble ligand has not yet been identified for HER2, which seems to be transactivated following heterodimerization. The heterodimerization of the erbB-2 receptor with the EGFR, HER3, and HER4 is preferred to homodimerization (Klapper et al., 1999; Klapper et al., 1997). Receptor dimerization results in binding of ATP to the receptor's catalytic site, activation of the receptor's tyrosine kinase, and autophosphorylation on C-terminal tyrosine residues. The phosphorylated tyrosine residues then serve as docking sites for proteins such as Grb2, Shc, and phospholipase C, that, in turn, activate downstream signaling pathways, including the Ras/MEK/Erk and the PI3K/Akt pathways (see FIG. 7), which regulate transcription factors and other proteins involved in biological responses such as proliferation, cell motility, angiogenesis, cell survival, and differentiation (Alroy & Yarden, 1997; Burgering & Coffer, 1995; Chan et al., 1999; Lewis et al., 1998; Liu et al., 1999; Muthuswamy et al., 1999; Riese & Stern, 1998).

ErbB-mediated activation of Akt requires the activation of PI3K (Knuefermann et al., 2003). This can occur via dimerization of ErbB2 or EGFR with HER3, which is able to couple to PI3K directly (Fedi et al., 1994), or by interaction of the receptor with the intracellular adaptor Gab1 (Rodrigues et al., 2000). Upon activation, PI3K converts phosphatidylinositol-4,5 bisphosphate (PIP2) to phosphatidylinositol-3,4,5 trisphosphate (PIP3); this lipid recruits the pleckstrin-homology (PH) domain of Akt to the plasma membrane where its kinase domain is activated (Chan et al., 1999). Akt, or protein kinase B, is a well-characterized serine/threonine kinase that promotes cellular survival and has three isoforms, Akt1, Akt2, and Akt3. Activation of all three isoforms is similar in that phosphorylation of two sites, one in the activation domain and one in the COOH-terminal hydrophobic motif, are necessary for full activity. For Akt1, phosphorylation of T308 in the activation domain by phosphoinositide-dependent kinase 1 is dependent on the products of PI3-K. Cellular levels of PIP₂ and PIP₃ are controlled by the tumor suppressor, dual-phosphatase PTEN, that dephosphorylates PIP₂ and PIP₃ at the 3′ position.

Once activated, Akt can suppress apoptosis by interacting with and phosphorylating several key downstream effectors. For example, Akt phosphorylates the proapoptotic Bcl-2 partner Bad, that binds to and blocks the activity of Bcl-x, a cell survival factor (del Peso et al., 1997); inactivates the initiation caspase-9 (Cardone et al., 1998); represses the forkhead transcription factor FKHRL-1 (Brunet et al., 1999), a regulator of the expression of the apoptosis-inducing Fas ligand; and phosphorylates IκB, promoting degradation of IκB and thereby increasing the activity of NFκB, a well-known cell survival factor (Ozes et al., 1999; Romashkova & Makarov, 1999). In addition to these molecules that are known to be involved in apoptosis, an increasing number of substrates involved in cell cycle regulation, protein synthesis, and glycogen metabolism are also phosphorylated by Akt (see the recent review by (Nicholson & Anderson, 2002)).

The MAP kinases ERK1 and ERK2 represent a central group of signaling kinases that are activated in response to ErbB signaling (for review see (Chang & Karin, 2001)). The best understood mechanism for activation of ERK is via growth factor receptor or tyrosine kinase activation of Ras. ERK has been implicated in the phosphorylation of a number of transcription factors that are important for expression of genes essential for cell proliferation (Chang & Karin, 2001). The mechanism by which ERK protects cells from apoptosis is complex, and Ras, a potent ERK activator, may also promote apoptosis (Kauffmann-Zeh et al., 1997). In cerebellar granular cells, ERK activation by survival factors prevents apoptosis through RSK, which inactivates the pro-apoptotic protein Bad (Bonni et al., 1999). ERK may also induce growth factors that promote cell survival.

Several strategies including monoclonal antibodies (Mab), immunoconjugates, anti-EGF vaccine, and tyrosine kinase inhibitors have been developed to target the ErbB family receptors and block their activation in cancer cells (reviewed in (Sridhar et al., 2003)). Because ErbB2-containing heterodimers are the most stable and preferred initiating event for signaling, interrupting both ErbB2 and EGFR simultaneously is an appealing therapeutic strategy. A series of quinazoline dual ErbB-2/EGFR TK inhibitors that possess efficacy in pre-clinical models for cancer have been synthesized (Cockerill et al., 2001; Rusnak et al., 2001a; Rusnak et al., 2001b). GW572016 is a quinazoline, orally active, reversible dual kinase inhibitor of both EGFR and ErbB2 kinases (Rusnak et al., 2001b). In human xenograft studies, GW572016 has shown dose-dependent kinase inhibition, and selectively inhibits tumor cells overexpressing EGFR or ErbB2 (Rusnak et al., 2001b; Xia et al., 2002).

The present inventors hypothesize that inhibition of both Akt kinase and Erk1/2 MAP kinases is required for the optimal induction of apoptosis of tumor cells by GW572016. It was further thought that the addition of an Akt kinase inhibitor to tumors in which GW572016 primarily causes reversible growth inhibition through Erk1/2 MAP kinases would augment the ability of GW572016 to induce cell death, a clinically desirable response. It was thought that a combination of an Akt kinase inhibitor and GW572016 or another inhibitor of ErbB signaling would produce synergistic apoptosis. These findings have implications for clinical applications of GW572016 where tumor regressions due to tumor cell death or apoptosis would be preferred. Consequently, it has now been recognized, that a combination of an erb family and PI3K and/or Akt inhibitors appears to be more effective than either therapy by itself. Accordingly, the present inventors have now discovered a new method of treating cancer using a novel pharmaceutical combination, which can selectively treat susceptible cancers. Specifically, the novel combination of a dual EGFR/erbB-2 inhibitor and a PI3K and/or Akt inhibitor appears to effectively inhibit growth of such tumors and at times the combination of a dual EGFR/erbB-2 inhibitor and a PI3K and/or Akt inhibitor may act synergistically.

### SUMMARY OF THE INVENTION

In a first aspect of the present invention, there is provided a method of treating a susceptible cancer in a mammal, comprising: administering to said mammal therapeutically effective amounts of (i) at least one erb family inhibitor and (ii) at least one of a PI3K and an Akt inhibitor.

In a second aspect of the present invention, there is provided a method of treating a susceptible cancer in a mammal, comprising: administering to said mammal therapeutically effective amounts of (i) a compound of formula (I) 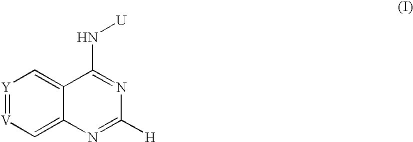
or a salt, solvate, physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino; U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 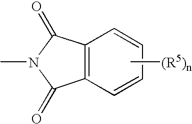
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one of a PI3K and an Akt inhibitor.

In a third aspect of the present invention, there is provided a method of treating a susceptible cancer in a mammal, comprising: administering to said mammal therapeutically effective amounts of (i) a compound of formula (II): 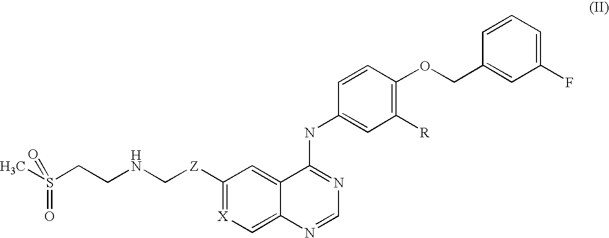
or salt or solvates thereof, wherein R is —Cl or —Br, X is CH, N, or CF, and Z is thiazole or furan; and
(ii) at least one of a PI3K and an Akt inhibitor.

In a fourth aspect of the present invention, there is provided a method of treating a susceptible cancer in a mammal, comprising: administering to said mammal therapeutically effective amounts of (i) a compound of formula (III): 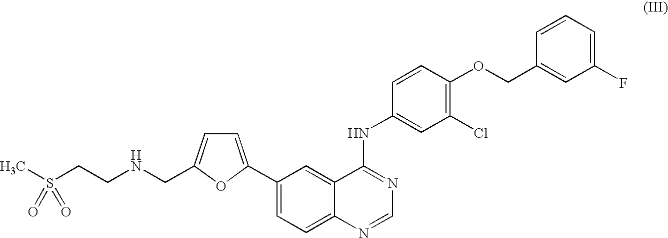
or salts or solvates thereof; and
(ii) at least one of a PI3K and an Akt inhibitor.

In a fifth aspect of the present invention, there is provided a cancer treatment combination, comprising: therapeutically effective amounts of (i) at least one erb family inhibitor and (ii) at least one of a PI3K and an Akt inhibitor.

In a sixth aspect of the present invention, there is provided a cancer treatment combination, comprising: therapeutically effective amounts of (i) a compound of formula (I) 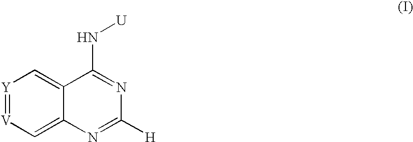
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 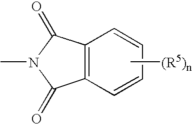
wherein each R⁵ is independently selected from halogen, C₁₋₄ alkyl and C₁₋₄ alkoxy; and n is 0 to 3;
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl; and
(ii) at least one of a PI3K and an Akt inhibitor.

In a seventh aspect of the present invention, there is provided a cancer treatment combination, comprising: therapeutically effective amounts of (i) a compound of formula (II): 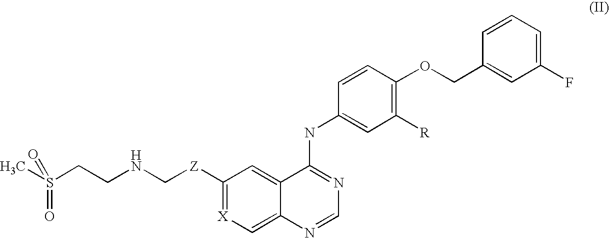
or salt or solvates thereof, wherein R is —Cl or —Br, X is CH, N, or CF, and Z is thiazole or furan; and
(ii) at least one of a PI3K and an Akt inhibitor.

In an eighth aspect of the present invention, there is provided a cancer treatment combination, comprising: therapeutically effective amounts of (i) a compound of formula (III): 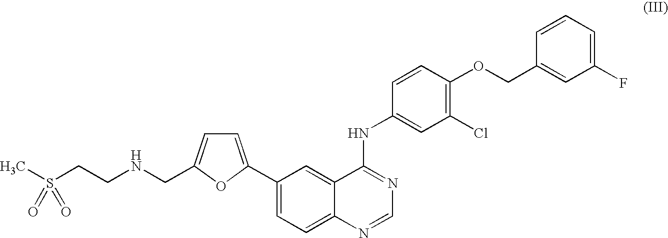
or salts or solvates thereof; and
(ii) at least one of a PI3K and an Akt inhibitor.

In a ninth aspect of the present invention, there is provided a cancer treatment combination, comprising: therapeutically effective amounts of (i) at least one erb family inhibitor and (ii) at least one of a PI3K and an Akt inhibitor for use in therapy.

In a tenth aspect of the present invention, there is provided a cancer treatment combination, comprising: therapeutically effective amounts of (i) at least one erb family inhibitor and (ii) at least one of a PI3K and an Akt inhibitor in the preparation of a medicament for use in the treatment of a susceptible cancer.

### BRIEF DESCRIPTION OF THE DRAWINGS

FIG. 1 depicts median effect analysis of 1:2 GW572016 and LY294002 in HN5 cells.

FIG. 2 depicts median effect analysis of 1:10 GW572016 and LY294002 in HN5 cells.

FIG. 3 depicts median effect analysis of 1:2 GW589522 and LY294002 in HN5 cells.

FIG. 4 depicts median effect analysis of 1:10 GW589522 and LY294002 in HN5 cells.

FIG. 5 depicts median effect analysis of 1:10 GW572016 and the compound of Example 9 in HN5 cells.

FIG. 6 depicts GW572016 and LY294002 synergistic action to induce apoptosis in T47D cells.

FIG. 7 depicts the PI3K/Akt pathway.

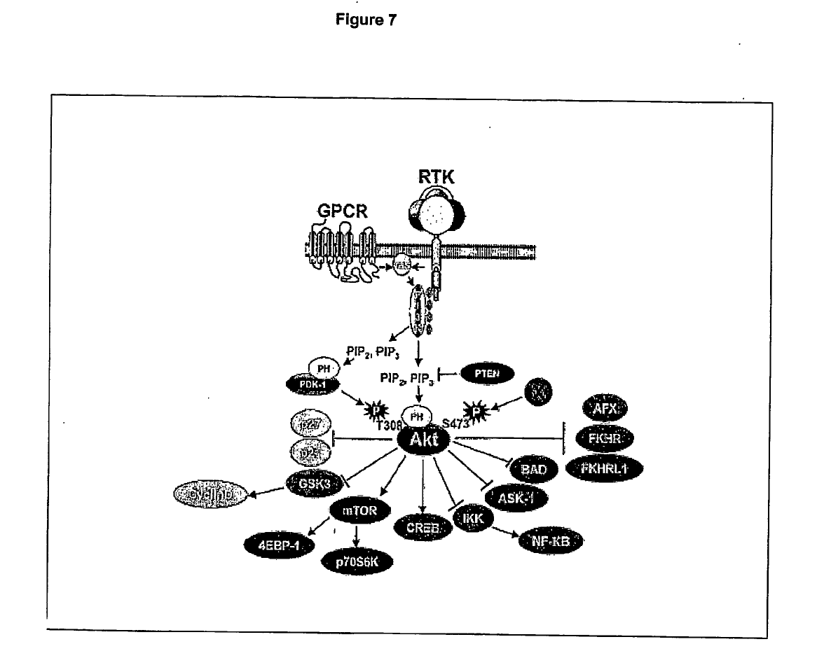

### DETAILED DESCRIPTION OF THE INVENTION

As used herein the term “neoplasm” refers to an abnormal growth of cells or tissue and is understood to include benign, i.e., non-cancerous growths, and malignant, i.e., cancerous growths. The term “neoplastic” means of or related to a neoplasm.

As used herein the term “agent” is understood to mean a substance that produces a desired effect in a tissue, system, animal, mammal, human, or other subject. Accordingly, the term “anti-neoplastic agent” is understood to mean a substance producing an anti-neoplastic effect in a tissue, system, animal, mammal, human, or other subject. It is also to be understood that an “agent” may be a single compound or a combination or composition of two or more compounds.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “Cₓ-C_{y}” or “C_{x-y}” where x and y represent an integer value refer to the number of carbon atoms in a particular chemical term to which it is attached. For instance, the term “C₁-C₄ alkyl” or “C₁₋₄ alkyl” refers to an alkyl group, as defined herein, containing at least 1, and at most 4 carbon atoms.

As used herein, the term “alkyl” refers to a straight or branched chain hydrocarbon radical having from one to twelve carbon atoms, optionally substituted with substituents selected from the group consisting of C₁-C₆ alkyl, C₁-C₆ hydroxyalkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aryl, aryloxy, heteroaryl, aminosulfonyl optionally substituted by alkyl, nitro, cyano, halogen, or C₁-C₆ perfluoroalkyl, multiple degrees of substitution being allowed. Examples of “alkyl” as used herein include, but are not limited to, methyl, ethyl, n-propyl, isopropyl, n-butyl, isobutyl, t-butyl, n-pentyl, isopentyl, and the like.

As used herein, the term “alkylene” refers to a straight or branched chain divalent hydrocarbon radical having from one to ten carbon atoms, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and C₁-C₆ perfluoroalkyl, multiple degrees of substitution being allowed. Examples of “alkylene” as used herein include, but are not limited to, methylene, ethylene, n-propylene, n-butylene, and the like.

As used herein, the term “alkenyl” refers to a hydrocarbon radical having from two to ten carbons and at least one carbon-carbon double bond, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen and C₁-C₆ perfluoroalkyl, multiple degrees of substitution being allowed. Examples of “alkenyl” as used herein include, ethenyl, propenyl, 1-butenyl, 2-butenyl, and isobutenyl.

As used herein, the term “alkynyl” refers to a hydrocarbon radical having from two to ten carbons and at least one carbon-carbon triple bond, optionally substituted with substituents selected from the group which includes C₁-C₆ alkyl, C₁-C₆ alkoxy, C₁-C₆ alkylsulfanyl, C₁-C₆ alkylsulfenyl, C₁-C₆ alkylsulfonyl, oxo, aryl, hydroxy, mercapto, amino optionally substituted by alkyl, carboxy, carbamoyl optionally substituted by alkyl, aminosulfonyl optionally substituted by alkyl, nitro, cyano, halogen and C₁-C₆ perfluoroalkyl, multiple degrees of substitution being allowed. Examples of “alkynyl” as used herein, include but are not limited to acetylenyl, 1-propynyl, 1-butynyl, 2-butynyl, 1-pentynyl, and 1-hexynyl.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I).

As used herein, the term “haloalkyl” refers to an alkyl group, as defined above, substituted with at least one halo group, halo being as defined herein. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo.

As used herein, the term “cycloalkyl” refers to a non-aromatic cyclic hydrocarbon ring, which optionally includes a C₁-C₆ alkyl linker through which it may be attached. Exemplary “cycloalkyl” groups useful in the present invention include, but are not limited to, cyclopropyl, cyclobutyl, cyclopentyl, cyclohexyl and cycloheptyl.

As used herein, the term “heterocyclic” or the term “heterocyclyl” refers to a three to twelve-membered non-aromatic heterocyclic ring, being saturated or having one or more degrees of unsaturation, containing one or more heteroatom substitutions selected from S, S(O), S(O)₂, O, or N, optionally substituted with substituents selected from the group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, carbamoyl optionally substituted by alkyl, aminosulfonyl optionally substituted by alkyl, nitro, cyano, halogen, or C₁-C₆ perfluoroalkyl, multiple degrees of substitution being allowed. Such a ring may be optionally fused to one or more other “heterocyclic” ring(s) or cycloalkyl ring(s). Examples of “heterocyclic” moieties include, but are not limited to, tetrahydrofuran, pyran, 1,4-dioxane, 1,3-dioxane, piperidine, piperazine, 2,4-piperazinedione, pyrrolidine, imidazolidine, pyrazolidine, morpholine, thiomorpholine, tetrahydrothiopyran, tetrahydrothiophene, and the like.

As used herein, the term “aryl” refers to an optionally substituted benzene ring or to an optionally substituted benzene ring system fused to one or more optionally substituted benzene rings to form, for example, anthracene, phenanthrene, or napthalene ring systems. Exemplary optional substituents include C₁-C₆ alkyl, C₁-C₆ alkoxy, C₁-C₆ haloalkyl, C₁-C₆ haloalkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino optionally substituted by alkyl or acyl, carboxy, tetrazolyl, carbamoyl optionally substituted by alkyl, aryl, or heteroaryl, aminosulfonyl optionally substituted by alkyl, acyl, aroyl, aroylamino, heteroaroyl, acyloxy, aroyloxy, heteroaroyloxy, alkoxycarbonyl, nitro, cyano, halogen, heteroaryl, heterocyclyl, aryl optionally substituted with aryl, halogen, C₁-C₆ alkyl, C₁-C₆ haloalkyl, or C₁-C₆ alkylsulfonyl, ureido, arylurea, alkylurea, cycloalkylurea, alkylthiourea, aryloxy, or aralkoxy, multiple degrees of substitution being allowed. Examples of “aryl” groups include, but are not limited to, phenyl, 2-naphthyl, 1-naphthyl, biphenyl, as well as substituted derivatives thereof.

As used herein, the term “aralkyl” refers to an aryl or heteroaryl group, as defined herein, attached through a C₁-C₃ alkylene linker, wherein the C₁-C₃ alkylene is as defined herein. Examples of “aralkyl” include, but are not limited to, benzyl, phenylpropyl, 2-pyridylmethyl, 3-isoxazolylmethyl, 5-methyl, 3-isoxazolylmethyl, and 2-imidazoyly ethyl.

As used herein, the term “heteroaryl” refers to a monocyclic five to seven membered aromatic ring, or to a fused bicyclic or tricyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of C₁-C₆ alkyl, C₁-C₆ alkoxy, C₁-C₆ alkylsulfanyl, C₁-C₆ alkylsulfenyl, C₁-C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, C₁-C₆ perfluoroalkyl, heteroaryl, or aryl, multiple degrees of substitution being allowed. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl, and substituted versions thereof.

As used herein, the term “alkoxy” refers to the group RₐO—, where Rₐ is alkyl as defined above. Exemplary alkoxy groups useful in the present invention include, but are not limited to, methoxy, ethoxy, n-propoxy, isopropoxy, n-butoxy, and t-butoxy.

As used herein, the term “amino” refers to the group —NH₂.

As used herein the term “alkylamino” refers to the group —NHRₐ wherein Rₐ is alkyl as defined above.

As used herein the term “arylamino” refers to the group —NHRₐ wherein Rₐ is aryl as defined above.

As used herein the term “aralkylamino” refers to the group —NHRₐ wherein Rₐ is an aralkyl group as defined above.

As used herein the term “aralkoxy” refers to the group R_{b}RₐO—, where Rₐ is alkyl and R_{b} is aryl or heteroaryl all as defined above.

As used herein the term “aryloxy” refers to the group RₐO—, where Rₐ is aryl or heteroaryl both as defined above.

As used herein the term “ureido” refers to the group —NHC(O)NH₂

As used herein, the term “arylurea” refers to the group —NHC(O)NHRₐ wherein Rₐ is aryl as defined above.

As used herein, the term “arylthiourea” refers to the group —NHC(S)NHRₐ wherein Rₐ is aryl as defined above.

As used herein, the term “alkylurea” refers to the group —NHC(O)NHRₐ wherein Rₐ is alkyl as defined above.

As used herein, the term “cycloalkylurea” refers to the group —NHC(O)NHRₐ wherein Rₐ is cycloalkyl as defined above.

As used herein, the term “cycloalkoxy” refers to the group RₐO—, where Rₐ is cycloalkyl as defined above. Exemplary cycloalkoxy groups useful in the present invention include, but are not limited to, cyclobutoxy, and cyclopentoxy.

As used herein, the term “haloalkoxy” refers to the group RₐO—, where Rₐ is haloalkyl as defined above. Exemplary haloalkoxy groups useful in the present invention include, but are not limited to, trifluoromethoxy.

As used herein, the terms “alkylsulfanyl” and “alkylthio” mean the same and refer to the group RₐS—, where Rₐ is alkyl as defined above.

As used herein, the term “haloalkylsulfanyl” refers to the group RₐS—, where Rₐ is haloalkyl as defined above.

As used herein, the term “alkylsulfenyl” refers to the group RₐS(O)—, where Rₐ is alkyl as defined above.

As used herein, the term “alkylsulfonyl” refers to the group RₐS(O)₂—, where Rₐ is alkyl as defined above.

As used herein, the term “alkylsulfonylamino” refers to the group —NHS(O)₂Rₐ wherein Ra is alkyl as defined above.

As used herein, the term “arylsulfonylamino” refers to the group —NHS(O)₂Rₐ wherein Ra is aryl as defined above.

As used herein, the term “alkylcarboxyamide” refers to the group —NHC(O)Rₐ wherein Rₐ is alkyl, amino, or amino substituted with alkyl, aryl or heteroaryl as described above.

As used herein, the term “oxo” refers to the group ═O.

As used herein, the term “mercapto” refers to the group —SH.

As used herein, the term “carboxy” refers to the group —C(O)OH.

As used herein, the term “cyano” refers to the group —CN.

As used herein the term “cyanoalkyl” refers to the group —CNRₐ, wherein Rₐ is alkyl as defined above. Exemplary “cyanoalkyl” groups useful in the present invention include, but are not limited to, cyanomethyl, cyanoethyl, and cyanoisopropyl.

As used herein, the term “aminosulfonyl” refers to the group —S(O)₂NH₂

As used herein, the term “carbamoyl” refers to the group —C(O)NH₂.

As used herein, the term “sulfanyl” shall refer to the group —S—.

As used herein, the term “sulfenyl” shall refer to the group —S(O)—.

As used herein, the term “sulfonyl” shall refer to the group —S(O)₂— or —SO₂—.

As used herein, the terms “acyl” and “alkylcarbonyl” are the same and refer to the group RₐC(O)—, where Rₐ is alkyl, cycloalkyl, or heterocyclyl as defined herein.

As used herein, the term “alkanoylamino” refers to the group RₐC(O)NH—, where Rₐ is alkyl as defined herein.

As used herein, the term “aroyl” refers to the group RₐC(O)—, where Rₐ is aryl as defined herein.

As used herein, the term “aroylamino” refers to the group RₐC(O)NH—, where Rₐ is aryl as defined herein.

As used herein, the term “heteroaroyl” refers to the group RₐC(O)—, where Rₐ is heteroaryl as defined herein.

As used herein, the term “alkoxycarbonyl” refers to the group RₐOC(O)—, where Rₐ is alkyl as defined herein.

As used herein, the term “acyloxy” refers to the group RₐC(O)O—, where Rₐ is alkyl, cycloalkyl, or heterocyclyl as defined herein.

As used herein, the term “aroyloxy” refers to the group RₐC(O)O—, where Rₐ is aryl as defined herein.

As used herein, the term “heteroaroyloxy” refers to the group RₐC(O)O—, where Rₐ is heteroaryl as defined herein.

As used herein, the term “optionally” means that the subsequently described event(s) may or may not occur, and includes both event(s), which occur, and events that do not occur.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention, a compounds formulae (I), (I′), (I^{a}), (I″), (II), (III), (III′), (III″) or (IV) or a salt or physiologically functional derivative thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

As used herein, the term “substituted” refers to substitution with the named substituent or substituents, multiple degrees of substitution being allowed unless otherwise stated.

Certain of the compounds described herein may contain one or more chiral atoms, or may otherwise be capable of existing as two enantiomers. The compounds of this invention include mixtures of enantiomers as well as purified enantiomers or enantiomerically enriched mixtures. Also included within the scope of the invention are the individual isomers of the compounds represented by formulae formulae (I), (I′), (I^{a}), (I″), (II), (III), (III′), (III″) or (IV) as well as any wholly or partially equilibrated mixtures thereof. The present invention also covers the individual isomers of the compounds represented by the formulas above as mixtures with isomers thereof in which one or more chiral centers are inverted. Also, it is understood that any tautomers and mixtures of tautomers of the compounds of formulae (I), (I′), (I^{a}), (I″), (II), (III), (III′), (III″) or (IV) are included within the scope of the compounds of formulae formulae (I), (I′), (Ia), (I″), (II), (III), (III′), (III″) or (IV).

As recited above, in one embodiment a method of treating cancer is provided which includes administering a therapeutically effective amount of at least one erb family inhibitor and at least one of a PI3K and an Akt inhibitor.

Preferably the erb family inhibitor is a dual inhibitor of erbB-2 and EGFR. Generally, any EGFR/erbB-2 inhibitor, that is any pharmaceutical agent having specific erbB-2 and/or EGFR inhibitor activity may be utilized in the present invention. Such erbB-2/EGFR inhibitors are described, for instance, in U.S. Pat. Nos. 5,773,476; 5,789,427; 6,103,728; 6,169,091; 6,174,889; and 6,207,669; and International Patent Applications WO 95/24190; WO 98/0234; WO 99/35146; WO 01/04111; and WO 02/02552 which patents and patent applications are herein incorporated by reference to the extent of their disclosure of erbB-2 and/or EGFR inhibitor compounds as well as methods of making the same.

In one embodiment of the present invention, the dual EGFR/erbB-2 inhibitor compounds are of the Formula I: 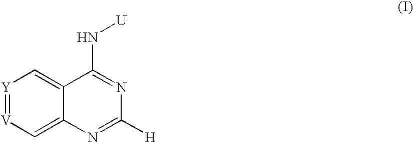
or a salt, solvate, or physiologically functional derivative thereof;
wherein
Y is CR¹ and V is N;
or Y is CR¹ and V is CR²;
R¹ represents a group CH₃SO₂CH₂CH₂NHCH₂—Ar—, wherein Ar is selected from phenyl, furan, thiophene, pyrrole and thiazole, each of which may optionally be substituted by one or two halo, C₁₋₄ alkyl or C₁₋₄ alkoxy groups;
R² is selected from the group comprising hydrogen, halo, hydroxy, C₁₋₄ alkyl, C₁₋₄ alkoxy, C₁₋₄ alkylamino and di[C₁₋₄ alkyl]amino;
U represents a phenyl, pyridyl, 3H-imidazolyl, indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group, substituted by an R³ group and optionally substituted by at least one independently selected R⁴ group;
R³ is selected from a group comprising benzyl, halo-, dihalo- and trihalobenzyl, benzoyl, pyridylmethyl, pyridylmethoxy, phenoxy, benzyloxy, halo-, dihalo- and trihalobenzyloxy and benzenesulphonyl;
or R³ represents trihalomethylbenzyl or trihalomethylbenzyloxy;
or R³ represents a group of formula 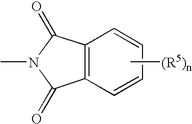
wherein each R⁵ is independently selected from halogen, C₁₋₄ alkyl and C₁₋₄ alkoxy; and n is 0 to 3; and
each R⁴ is independently hydroxy, halogen, C₁₋₄ alkyl, C₂₋₄ alkenyl, C₂₋₄ alkynyl, C₁₋₄ alkoxy, amino, C₁₋₄ alkylamino, di[C₁₋₄ alkyl]amino, C₁₋₄ alkylthio, C₁₋₄ alkylsulphinyl, C₁₋₄ alkylsulphonyl, C₁₋₄ alkylcarbonyl, carboxy, carbamoyl, C₁₋₄ alkoxycarbonyl, C₁₋₄ alkanoylamino, N—(C₁₋₄ alkyl)carbamoyl, N,N-di(C₁₋₄ alkyl)carbamoyl, cyano, nitro and trifluoromethyl.

The definitions for Y and V thus give rise to two possible basic ring systems for the compounds of formula (I). In particular the compounds may contain the following basic ring systems: quinazolines (1) and pyrido-pyrimidines (2):

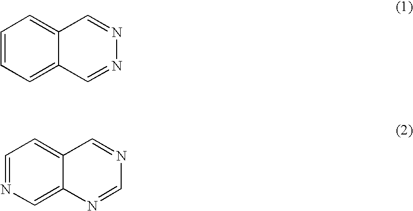

In a preferred embodiment, the ring system is ring (1).

Suitable values for the various groups listed above within the definitions for R¹, R², R⁴ and R⁵ are as follows:

halo is, for example, fluoro, chloro, bromo or iodo; preferably it is fluoro, chloro or bromo, more preferably fluoro or chloro;

C₁₋₄ alkyl is, for example, methyl, ethyl, propyl, isopropyl, butyl, isobutyl, sec-butyl or tert-butyl; preferably it is methyl, ethyl, propyl, isopropyl or butyl, more preferably methyl;

C₂₋₄ alkenyl is, for example, ethenyl, prop-1-enyl or prop-2-enyl; preferably ethenyl;

C₂₋₄ alkynyl is, for example, ethynyl, prop-1-ynyl or prop-2-ynyl; preferably ethynyl;

C₁₋₄ alkoxy is, for example, methoxy, ethoxy, n-propoxy, isopropoxy, n-butoxy, isobutoxy, sec-butoxy or tert-butoxy; preferably methoxy, ethoxy, propoxy, isopropoxy or butoxy; more preferably methoxy;

C₁₋₄ alkylamino is, for example, methylamino, ethylamino or propylamino; preferably methylamino;

di[C₁₋₄ alkyl]amino is, for example, dimethylamino, diethylamino, N-methyl-N-ethylamino or dipropylamino; preferably dimethylamino;

C₁₋₄ alkylthio is, for example, methylthio, ethylthio, propylthio or isopropylthio, preferably methylthio;

C₁₋₄ alkylsulphinyl is, for example, methylsulphinyl, ethylsulphinyl, propylsulphinyl or isopropylsulphinyl, preferably methylsulphinyl;

C₁₋₄ alkylsulphonyl is, for example, methanesulphonyl, ethylsulphonyl, propylsulphonyl or isopropylsulphonyl, preferably methanesulphonyl;

C₁₋₄ alkylcarbonyl is, for example methylcarbonyl, ethylcarbonyl or propylcarbonyl;

C₁₋₄ alkoxycarbonyl is, for example, methoxycarbonyl, ethoxycarbonyl, propoxycarbonyl, butoxycarbonyl or tert-butoxycarbonyl;

C₁₋₄ alkanoylamino (where the number of carbon atoms includes the CO functionality) is, for example, formamido, acetamido, propionamido or butyramido;

N—(C₁₋₄ alkyl)carbamoyl is, for example, N-methylcarbamoyl or N-ethylcarbamoyl; and

N,N-di(C₁₋₄ alkyl)carbamoyl is, for example, N,N-dimethylcarbamoyl, N-methyl-N-ethylcarbamoyl or N,N-diethylcarbamoyl.

In a preferred embodiment, Y is CR¹ and V is CR² (ring system (1) above).

In another embodiment, Y is CR¹ and V is N (ring system (2) above).

In one embodiment, R² represents hydrogen or C₁₋₄ alkoxy.

In a preferred embodiment, R² represents hydrogen or methoxy.

In another preferred embodiment, R² represents halo; more preferred R² is fluoro.

In a preferred embodiment, the group Ar is substituted by one halo, C₁₋₄ alkyl or C₁₋₄ alkoxy group.

In a more preferred embodiment, the group Ar is substituted by a C₁₋₄ alkyl group.

In another preferred embodiment, the group Ar does not carry any optional substituents.

In a further more preferred embodiment, Ar represents furan, phenyl or thiazole, each of which may optionally be substituted as indicated above.

In a further more preferred embodiment, Ar represents furan or thiazole, each of which may optionally be substituted as indicated above.

In a most preferred embodiment, Ar represents unsubstituted furan or thiazole.

The side chain CH₃SO₂CH₂CH₂NHCH₂ may be linked to any suitable position of the group Ar. Similarly, the group R¹ may be linked to the carbon atom carrying it from any suitable position of the group Ar.

In a preferred embodiment, when Ar represents furan the side chain CH₃SO₂CH₂CH₂NHCH₂ is in the 4-position of the furan ring and the link to the carbon atom carrying the group R¹ is from the 2-position of the furan ring.

In another preferred embodiment, when Ar represents furan the side chain CH₃SO₂CH₂CH₂NHCH₂ is in the 3-position of the furan ring and the link to the carbon atom carrying the group R¹ is from the 2-position of the furan ring.

In a most preferred embodiment, when Ar represents furan the side chain CH₃SO₂CH₂CH₂NHCH₂ is in the 5-position of the furan ring and the link to the carbon atom carrying the group R¹ is from the 2-position of the furan ring.

In a further most preferred embodiment, when Ar represents thiazole the side chain CH₃SO₂CH₂CH₂NHCH₂ is in the 2-position of the thiazole ring and the link to the carbon atom carrying the group R¹ is from the 4-position of the thiazole ring.

The R³ and R⁴ groups may be bound to the ring system U by either a carbon atom or a heteroatom of the ring system. The ring system itself may be bound to the bridging NH group by a carbon atom or a heteroatom but is preferably bound by a carbon atom. The R³ and R⁴ groups may be bound to either ring when U represents a bicyclic ring system, but these groups are preferably bound to the ring which is not bound to the bridging NH group in such a case.

In a preferred embodiment U, represents a phenyl, indolyl, or 1H-indazolyl group substituted by an R³ group and optionally substituted by at least one independently selected R⁴ group.

In a more preferred embodiment, U represents a phenyl or 1H-indazolyl group substituted by an R³ group and optionally substituted by at least one independently selected R⁴ group.

In a more preferred embodiment, where U represents a phenyl group the group R³ is in the para-position relative to the bond from U to the linking NH group.

In a further more preferred embodiment, where U represents a 1H-indazolyl group the group R³ is in the 1-position of the indazolyl group.

In a preferred embodiment, R³ represents benzyl, pyridylmethyl, phenoxy, benzyloxy, halo-, dihalo- and trihalobenzyloxy and benzenesulphonyl.

In a further preferred embodiment, R³ represents trihalomethylbenzyloxy.

In a further preferred embodiment, R³ represents a group of formula 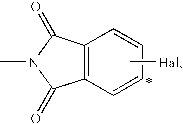
wherein Hal is Br or Cl, particularly Cl, more especially wherein the Hal substituent is in the position marked with a star in the ring as shown.

In a more preferred embodiment, R³ represents benzyloxy, fluorobenzyloxy (especially 3-fluorobenzyloxy), benzyl, phenoxy and benzenesulphonyl.

In a further more preferred, embodiment R³ represents bromobenzyloxy (especially 3-bromobenzyloxy) and trifluoromethylbenzyloxy.

In a further preferred embodiment, the ring U is not substituted by an R⁴ group; in an especially preferred embodiment U is phenyl or indazolyl unsubstituted by an R⁴ group.

In a further preferred embodiment, the ring U is substituted by an R⁴ group selected from halo or C₁₋₄ alkoxy; especially chloro, fluoro or methoxy.

In a more preferred embodiment, the ring U is substituted by an R⁴ group wherein R⁴ represents halo, especially 3-fluoro.

In another preferred embodiment, U together with R⁴ represents methoxyphenyl, fluorophenyl, trifluoromethylphenyl or chlorophenyl.

In a further preferred embodiment, U together with R⁴ represents methoxyphenyl or fluorophenyl.

In another preferred embodiment, the group U together with the substituent(s) R³ and R⁴ represents benzyloxyphenyl, (fluorobenzyloxy)phenyl, (benzenesulphonyl)phenyl, benzylindazolyl or phenoxyphenyl.

In still another preferred embodiment, the group U together with the substituent(s) R³ and R⁴ represents benzyloxyphenyl, (3-fluorobenzyloxy)phenyl, (benzenesulphonyl)phenyl or benzylindazolyl.

In another preferred embodiment, the group U together with the substituent(s) R³ and R⁴ represents (3-bromobenzyloxy)phenyl, (3-trifluoromethylbenzyloxy)phenyl, or (3-fluorobenzyloxy)-3-methoxyphenyl.

In a more preferred embodiment, the group U together with the substituent(s) R³ and R⁴ represents 3-fluorobenzyloxy-3-chlorophenyl, benzyloxy-3-chlorophenyl, benzyloxy-3-trifluoromethylphenyl, (benzyloxy)-3-fluorophenyl, (3-fluorobenzyloxy)-3-fluorophenyl or (3-fluorobenzyl)indazolyl.

In another preferred embodiment the group U together with the substituent(s) R³ and R⁴ represents benzyloxyphenyl or (3-fluorobenzyloxy)phenyl.

In a preferred embodiment, there is provided a compound of formula (I) or a salt, solvate, or physiologically functional derivative thereof wherein V is CR², wherein R² is hydrogen, halo (especially fluoro) or C₁₋₄ alkoxy (especially methoxy); Y is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (especially chloro or fluoro), or methoxy.

In another preferred embodiment, there is provided a compound of formula (i) or a salt, solvate, or physiologically functional derivative thereof wherein V is CR², wherein R² is hydrogen, halo (especially fluoro) or C₁₋₄ alkoxy (especially methoxy); Y is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (especially chloro or fluoro), or methoxy.

In a preferred embodiment, there is provided a compound of formula (I) or a salt, solvate, or physiologically functional derivative thereof wherein V is CR², wherein R² is hydrogen, halo (especially fluoro) or C₁₋₄ alkoxy (especially methoxy); Y is CR¹ wherein R¹ is as defined above in which Ar is unsubstituted furan or thiazole; U is indazole; R³ is benzyl or fluorobenzyl; and R⁴ is not present.

In a further preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (especially chloro or fluoro), or methoxy.

In a another preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (especially chloro or fluoro), or methoxy.

In another preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted furan or thiazole; U is indazole; R³ is benzyl or fluorobenzyl; and R⁴ is not present.

In another preferred embodiment, there is provided a compound of formula (I) or a salt, solvate, or physiologically functional derivative thereof wherein Y is CR², wherein R² is hydrogen, halo (especially fluoro) or C₁₋₄ alkoxy (especially methoxy); V is CR¹ wherein R¹ is as defined above in which Ar is unsubstituted furan or thiazole; U is phenyl; R³ is phenoxy; and R⁴ is not present.

In another more preferred embodiment, there is provided a compound of formula (I) or a salt, solvate, or physiologically functional derivative thereof wherein V is N; Y is CR¹ wherein R¹ is as defined above in which Ar is unsubstituted phenyl, furan or thiazole; U is phenyl or indazole; R³ is benzyl, fluorobenzyl, benzyloxy, fluorobenzyloxy, bromobenzyloxy, trifluoromethylbenzyloxy, phenoxy or benzenesulphonyl; and R⁴ is not present or is halo (especially chloro or fluoro), or methoxy.

In another most preferred embodiment, there is provided a compound of formula (I) or a salt, solvate, or physiologically functional derivative thereof wherein V is N, Y is CR¹ wherein R¹ is as defined above in which Ar is unsubstituted furan or thiazole; U is phenyl; R³ is benzyloxy, fluorobenzyloxy or benzenesulphonyl; and R⁴ is not present or is halo (especially chloro or fluoro), or methoxy.

In another most preferred embodiment, there is provided a compound of formula (I) or a salt, solvate, or physiologically functional derivative thereof wherein V is N, Y is CR¹ wherein R¹ is as defined above in which Ar is unsubstituted furan or thiazole; U is indazole; R³ is benzyl or fluorobenzyl; and R⁴ is not present.

In another embodiment, the compound of formula (I) is a compound of formula (II):

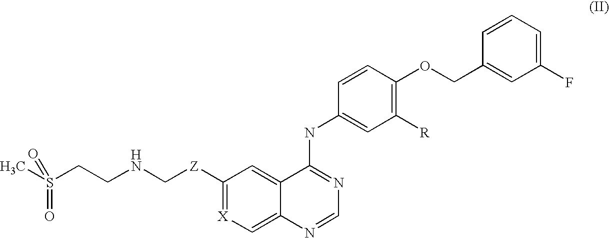

or salt or solvate thereof, wherein R is —Cl or —Br, X is CH, N, or CF, and Z is thiazole or furan.

In another embodiment, the compound of formula (I) is a compound of formula (III): 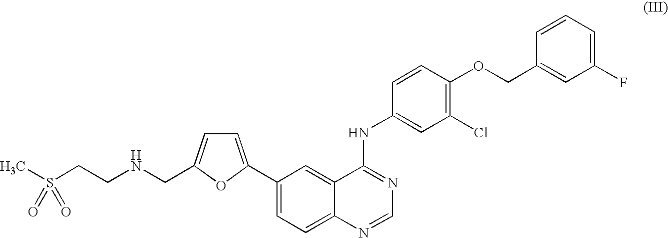
or salts or solvates thereof.

In another embodiment, the compound of formula (I) is a ditosylate salt of the compound of formula (III) and anhydrate or hydrate forms thereof. The ditosylate salt of the compound of formula (III) has the chemical name N-{3-chloro-4-[(3-fluorobenzyl)oxy]phenyl}-6-[5-({[2-(methanesulphonyl)ethyl]amino}methyl)-2-furyl]-4-quinazolinamine ditosylate. In one embodiment, the compound of formula (I) is the anhydrous ditosylate salt of the compound of formula (III). In another embodiment, the compound of formula (I) is the monohydrate ditosylate salt of the compound of formula (III).

In another embodiment, the compound of formula (I) is a compound of formula (II) wherein, R is Cl; X is CH; and Z is thiazole. In a preferred embodiment, the compound of formula (I) is a ditosylate salt of a compound of formula (II) wherein, R is Cl; X is CH; and Z is thiazole; and anhydrate or hydrate forms thereof. The chemical name of such compound of formula (II) is (4-(3-fluoro-benzyloxy)-3-chlorophenyl)-(6-(2-((2-methanesulphonyl-ethylamino)-methyl)-thiazol-4-yl)quinazolin-4-yl)-amine and is a compound of formula (III′).

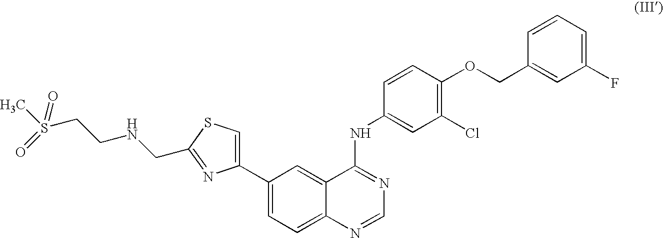

In another embodiment, the compound of formula (I) is a compound of formula (II) wherein, R is Br; X is CH; and Z is furan. In a preferred embodiment, the compound of formula (I) is a ditosylate salt of the compound of formula (II) wherein, R is Br; X is CH; and Z is furan; and anhydrate or hydrate forms thereof. The chemical name of such compound of formula (II) is (4-(3-fluoro-benzyloxy)-3-bromophenyl)-(6-(5-((2-methanesulphonyl-ethylamino)-methyl)-furan-2-yl)quinazolin-4-yl)-amine and is a compound of formula (III″).

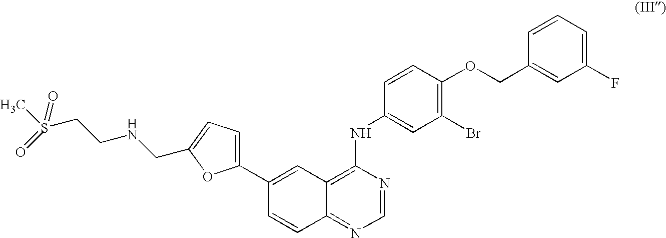

The free base, HCl salts, and ditosylate salts of the compounds of Formulae (I), (II), (III), (III′) and (III″) may be prepared according to the procedures of International Patent Application No. PCT/EP99/00048, filed Jan. 8, 1999, and published as WO 99/35146 on Jul. 15, 1999, referred to above and International Patent Application No. PCT/US01/20706, filed Jun. 28, 2001 and published as WO 02/02552 on Jan. 10, 2002 and according to the appropriate Examples recited below. One such procedure for preparing the ditosylate salt of the compound of formula (III) is presented following in Scheme 1.

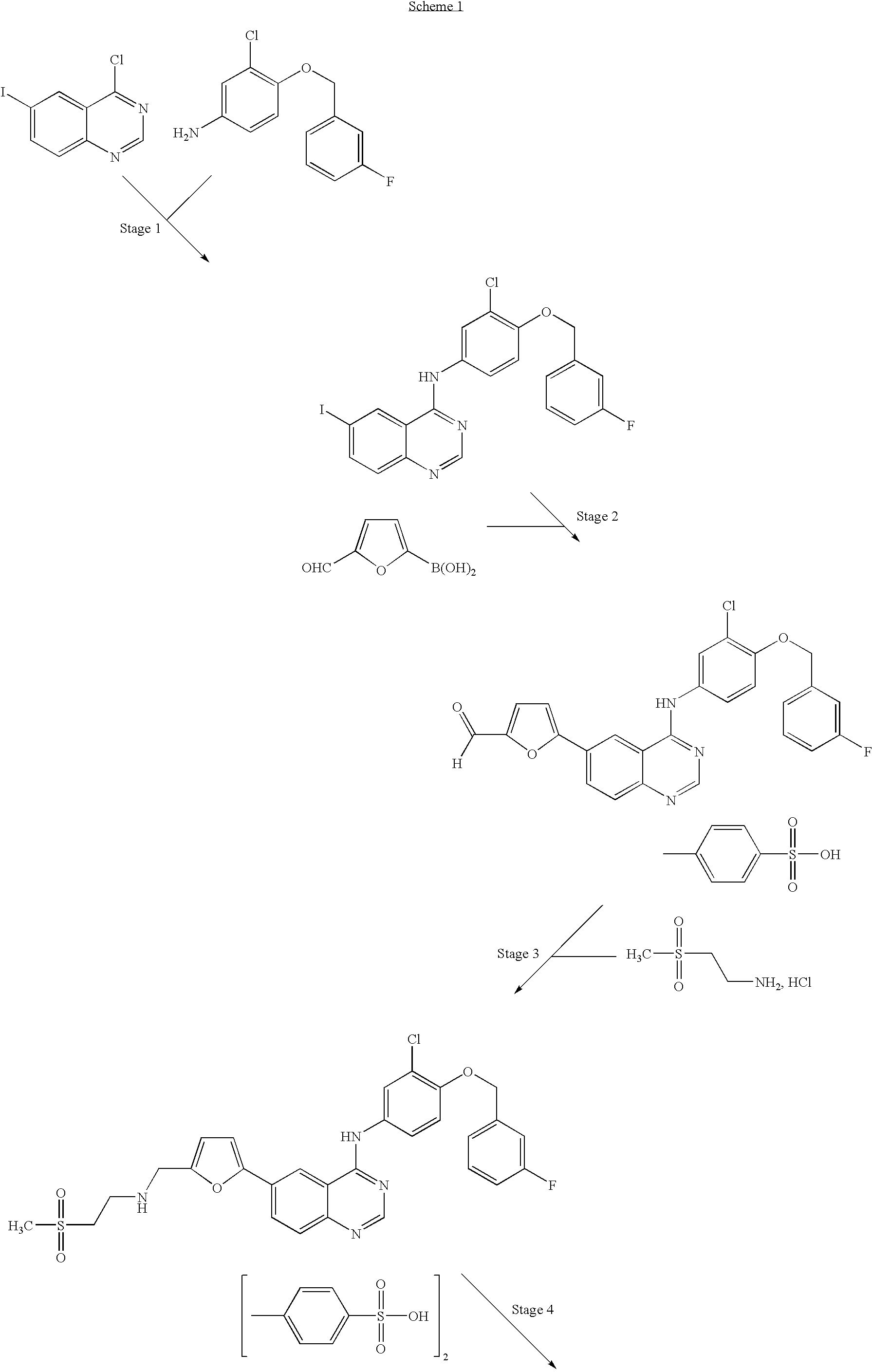

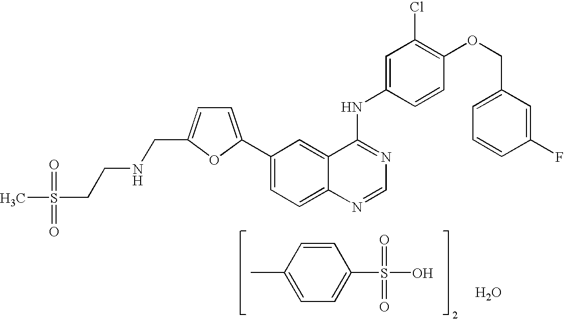

In scheme 1, the preparation of the ditosylate salt of the compound of formula (III) proceeds in four stages: Stage 1: Reaction of the indicated bicyclic compound and amine to give the indicated iodoquinazoline derivative; Stage 2: preparation of the corresponding aldehyde salt; Stage 3: preparation of the quinazoline ditosylate salt; and Stage 4: monohydrate ditosylate salt preparation.

In another embodiment of the present invention, the EGFR/erbB-2 inhibitor compounds are compounds of the Formula I′: 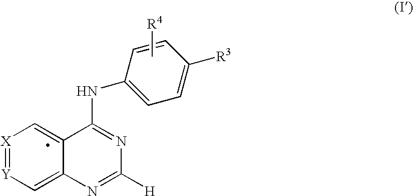
or a salt, solvate, or a physiologically functional derivative thereof;
wherein
X is CR¹ and Y is N;
or X is CR¹ and Y is CR²;
R¹ represents a group R⁵SO₂CH₂CH₂Z-(CH₂)ₚ—Ar—, wherein Ar is selected from phenyl, furan, thiophene, pyrrole and thiazole, each of which may optionally be substituted by one or two halo, C₁₋₄ alkyl or C₁₋₄ alkoxy groups; Z represents O, S, NH or NR⁶; p is 1, 2, 3 or 4;
R⁵ is C₁₋₆ alkyl optionally substituted by one or more R⁸ groups;
or R⁵ is C₁₋₆ alkyl substituted by a group Het or a group Cbc, each of which may be optionally substituted by one or more R⁸ groups;
or R⁵ is selected from a group Het or a group Cbc, each of which may be optionally substituted by one or more R⁸ groups;
each R⁸ is independently selected from halo, hydroxy, C₁₋₄ alkoxy, nitrile, NH₂ or NR⁶R⁷;
R⁶ is C₁₋₄alkyl, C₁₋₄alkoxy-C₁₋₄alkyl, hydroxyC₁₋₄alkyl, CF₃C(O) or CH₃C(O);
R⁷ is hydrogen or R⁶;
R² is selected from hydrogen, halo, hydroxy, C₁₋₄ alkyl or C₁₋₄ alkoxy;
R³ is selected from pyridylmethoxy, benzyloxy, halo-, dihalo- or trihalobenzyloxy; and
R⁴ is selected from hydrogen, halogen, C₁₋₄ alkyl, C₂₋₄ alkynyl or cyano.

In a preferred embodiment, R⁴ is located on the phenyl ring as indicated in formula (I^{a}).

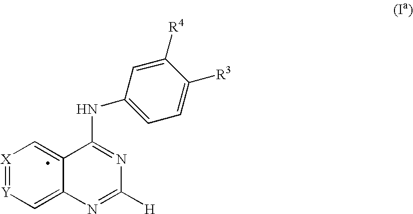

In one embodiment, the group R⁵ is an alkylene group linked to a Het or Cbc group, the alkylene group is preferably C₁₋₄ alkylene, more preferably C₁₋₃ alkylene, most preferably methylene or ethylene.

The definitions for X and Y thus give rise to two possible basic ring systems for the compounds of formula (I′). In particular the compounds may contain the following basic ring systems: quinazolines (1) and pyrido-pyrimidines (2)

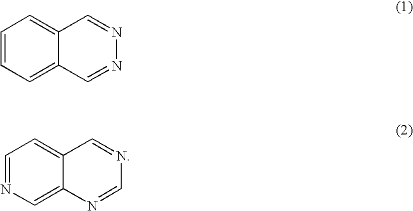

Ring system (1) is preferred.

The group Het comprise one or more rings which may be saturated, unsaturated, or aromatic and which may independently contain one or more nitrogen, oxygen, or sulfur heteroatoms, where N-oxides and sulfur monoxides and sulfur dioxides are permissible heteroaromatic substitutions in each ring.

Examples of suitable Het groups include acridine, benzimidazole, benzofuran, benzothiophene, benzoxazole, benzthiazole, carbazole, cinnoline, dioxin, dioxane, dioxalane, dithiane, dithiazine, dithiazole, dithiolane, furan, imidazole, imidazoline, imidazolidine, indole, indoline, indolizine, indazole, isoindole, isoquinoline, isoxazole, isothiazole, morpholine, napthyridine, oxazole, oxadiazole, oxathiazole, oxathiazolidine, oxazine, oxadiazine, phenazine, phenothiazine, phenoxazine, phthalazine, piperazine, piperidine, pteridine, purine, pyran, pyrazine, pyrazole, pyrazoline, pyrazolidine, pyridazine, pyridine, pyrimidine, pyrrole, pyrrolidine, pyrroline, quinoline, quinoxaline, quinazoline, quinolizine, tetrahydrofuran, tetrazine, tetrazole, thiophene, thiadiazine, thiadiazole, thiatriazole, thiazine, thiazole, thiomorpholine, thianaphthalene, thiopyran, triazine, triazole, or trithiane.

Preferred Het groups are aromatic groups selected from furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole.

More preferred Het groups are aromatic groups selected from furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine.

Most preferred Het groups are aromatic groups selected from pyridine and imidazole, especially pyrid-2-yl and imidazol-2-yl.

Cbc groups comprise one or more rings which may be independently saturated, unsaturated, or aromatic and which contain only carbon and hydrogen.

Preferred Cbc groups include aromatic groups selected from phenyl, biphenyl, naphthyl (including 1-naphthyl and 2-naphthyl) and indenyl.

Further suitable Cbc groups include cyclopropyl, cyclobutyl, cyclopentyl, cyclohexyl, cycloheptyl, cyclooctyl, tetralin, decalin, cyclopentenyl and cyclohexenyl.

A more preferred Cbc group is phenyl.

In one embodiment, Het groups and Cbc groups included within the group R⁵ are unsubstituted.

In a preferred embodiment, X is CR¹ and Y is CR² (ring system (1) above).

In a further preferred embodiment, X is CR¹ and Y is N (ring system (2) above.

In a preferred embodiment, R² represents hydrogen, halogen or C₁₋₄ alkoxy. In a more preferred embodiment R² represents hydrogen, fluoro or methoxy. In a most preferred embodiment R² represents hydrogen or fluoro.

In a preferred embodiment, Z represents NH, NR⁶ or O. In a more preferred embodiment Z presents NH or O. In a most preferred embodiment Z represents NH.

In a preferred embodiment, p is 1, 2 or 3.

In a further preferred embodiment, the group Ar does not carry any optional substituents.

In a further preferred embodiment, Ar represents furan or thiazole.

In a preferred embodiment, R⁵ represents an aromatic Het or Cbc group optionally substituted by a C₁₋₄ alkyl group (especially a methyl group).

In a more preferred embodiment, R⁵ represents pyridyl (especially pyrid-2-yl), phenyl, imidazolyl or N-methylimidazolyl (especially imidazol-2-yl).

In a preferred embodiment, R⁵ represents C₁₋₆ alkyl optionally substituted by one or more groups selected from halo, hydroxy, C₁₋₄ alkoxy, nitrile, NH₂ or NR⁶R⁷, wherein R⁷ represents H or R⁶, wherein R⁶ is as defined above.

In a more preferred embodiment, R⁵ represents C₁₋₆alkyl optionally substituted by one or more groups selected from hydroxy, C₁₋₄ alkoxy, NH₂ or NR⁶R⁷, wherein R⁷ represents H or R⁶; and R⁶ represents C₁₋₄ alkyl.

In a most preferred embodiment, R⁵ represents unsubstituted C₁₋₆alkyl; especially unsubstituted C₁₋₄ alkyl.

The side chain R⁵SO₂CH₂CH₂Z-(CH₂)ₚ may be linked to any suitable position of the group Ar. Similarly, the group R¹ may be linked to the carbon atom carrying it from any suitable position of the group Ar.

In a more preferred embodiment, when Ar represents furan the side chain R⁵SO₂CH₂CH₂Z-(CH₂)ₚ is in the 5-position of the furan ring and the link to the carbon atom carrying the group R¹ is from the 2-position of the furan ring.

In a further more preferred embodiment, when Ar represents thiazole the side chain R⁵SO₂CH₂CH₂Z-(CH₂)ₚ is in the 2-position of the thiazole ring and the link to the carbon atom carrying the group R¹ is from the 4-position of the thiazole ring.

In a preferred embodiment, R³ represents benzyloxy or fluorobenzyloxy (especially 3-fluorobenzyloxy).

In an especially preferred embodiment, R⁴ represents chloro, bromo, or hydrogen.

In a most especially preferred embodiment, R³ is represents benzyloxy or 3-fluorobenzyloxy and R⁴ chloro or bromo.

In a more preferred embodiment, there is provided a compound of formula (I′) or a salt, solvate or physiologically functional derivative thereof wherein Y is CR², wherein R² is hydrogen, fluoro or methoxy; X is CR¹ wherein R¹ is as defined above in which Ar is unsubstituted furan or thiazole; R³ is benzyloxy or fluorobenzyloxy; and R⁴ is hydrogen, or is chloro or bromo.

In a further more preferred embodiment, there is provided a compound of formula (I′) or a salt or solvate thereof wherein Y is N; X is CR¹ wherein R¹ is as defined above in which Ar is unsubstituted furan or thiazole; R³ is benzyloxy or fluorobenzyloxy; and R⁴ is hydrogen, or is chloro or bromo.

In a most preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is fluorobenzyloxy; and R⁴ is chloro or bromo.

In a further most preferred embodiment, there is provided a compound of formula (I′) or a salt or solvate thereof wherein Y is N; X is CR¹ wherein R¹ is as defined above in which Ar is unsubstituted furan or thiazole; R³ is fluorobenzyloxy; and R⁴ is chloro or bromo.

In a more preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is benzyloxy or fluorobenzyloxy; R⁴ is hydrogen, or is chloro or bromo; and R⁵ is unsubstituted C₁ alkyl.

In a further more preferred embodiment, there is provided a compound of formula (I′) or a salt, solvate or physiologically functional derivative thereof wherein Y is N; X is CR¹ wherein R¹ is as defined above in which Ar is unsubstituted furan or thiazole; R³ is benzyloxy or fluorobenzyloxy; R⁴ is hydrogen, or is chloro or bromo; and R⁵ is unsubstituted C₁₋₆ alkyl.

In a most preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is fluorobenzyloxy; R⁴ is chloro or bromo; and R⁵ is unsubstituted C₁₋₆ alkyl.

In a further most preferred embodiment, there is provided a compound of formula (I′) or a salt or solvate thereof wherein Y is N; X is CR¹ wherein R¹ is as defined above in which Ar is unsubstituted furan or thiazole; R³ is fluorobenzyloxy; R⁴ is chloro or bromo; and R⁵ is unsubstituted C₁₋₆ alkyl.

In a more preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is benzyloxy or fluorobenzyloxy; R⁴ is hydrogen, or is chloro or bromo; and R⁵ is pyridine, imidazole, or phenyl.

In a further more preferred embodiment, there is provided a compound of formula (I′) or a salt, solvate or physiologically functional derivative thereof wherein Y is N; X is CR¹ wherein R¹ is as defined above in which Ar is unsubstituted furan or thiazole; R³ is benzyloxy or fluorobenzyloxy; R⁴ is hydrogen, or is chloro or bromo; and R⁵ is pyridine, imidazole, or phenyl.

In a most preferred embodiment, there is provided a compound of formula (I′) or a salt or solvate thereof wherein Y is CR², wherein R² is hydrogen, fluoro or methoxy; X is CR¹ wherein R¹ is as defined above in which Ar is unsubstituted furan or thiazole; R³ is fluorobenzyloxy; R⁴ is chloro or bromo; and R⁵ is pyridine, imidazole, or phenyl.

In a further most preferred embodiment there is provided a compound of formula (I′) or a salt or solvate thereof wherein Y is N; X is CR¹ wherein R¹ is as defined above in which Ar is unsubstituted furan or thiazole; R³ is fluorobenzyloxy; R⁴ is chloro or bromo; and R⁵ is pyridine, imidazole, or phenyl.

A group of preferred species of compounds of Formula (I′) are:

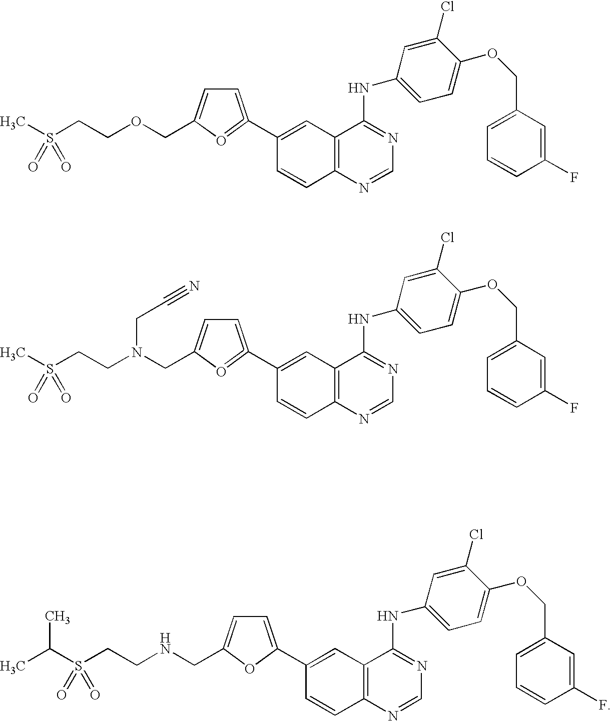

The compounds of Formulae (I′) and (1^{a}) may be prepared according to the procedures of International Patent Application No. PCT/US00/18128, filed Jun. 30, 2000, and published as WO 01/04111 on Jan. 18, 2001, referred to above and according to the appropriate Examples recited below.

In a further embodiment of the present invention, the dual EGFR/erbB-2 inhibitor compounds are compounds of the Formula I″: 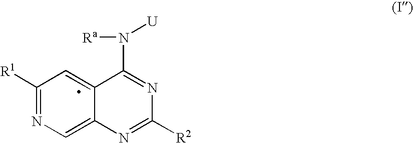
or a salt, solvate, or physiologically functional derivative thereof;
wherein

R^{a} is hydrogen or a C₁₋₈ alkyl group

R¹ is independently selected from the group comprising amino, hydrogen, halo, hydroxy, nitro, carboxy, formyl, cyano, trifluoromethyl, trifluoromethoxy, carbamoyl, ureido, guanidino, C₁₋₈ alkyl, C₁₋₈ alkoxy, C₃₋₈ cycloalkoxy, C₄₋₈ alkylcycloalkoxy, C₁₋₈ alkylcarbonyl, C₁₋₈ alkoxycarbonyl, N—C₁₋₄ alkylcarbamoyl, N,N-di-[C₁₋₄ alkyl]carbamoyl, hydroxyamino, C₁₋₄ alkoxyamino, C₂₋₄ alkanoyloxyamino, C₁₋₄ alkylamino, di[C₁₋₄ alkyl]amino, di-[C₁₋₄ alkyl]amino-C₁₋₄ alkylene-(C₁₋₄ alkyl)amino, C₁₋₄ alkylamino-C₁₋₄ alkylene-(C₁₋₄ alkyl)amino, hydroxy-C₁₋₄ alkylene-(C₁₋₄ alkyl)amino, phenyl, phenoxy, 4-pyridon-1-yl, pyrrolidin-1-yl, imidazol-1-yl, piperidino, morpholino, thiomorpholino, thiomorpholino-1-oxide, thiomorpholino-1,1-dioxide, piperazin-1-yl, 4-C₁₋₄ alkylpiperazin-1-yl, dioxolanyl, C₁₋₈ alkylthio, arylthio, C₁₋₄ alkylsulphinyl, C₁₋₄ alkylsulphonyl, arylsulphinyl, arylsulphonyl, halogeno-C₁₋₄ alkyl, hydroxy-C₁₋₄ alkyl, C₂₋₄ alkanoyloxy-C₁₋₄ alkyl, C₁₋₄ alkoxy-C₁₋₄ alkyl, carboxy-C₁₋₄ alkyl, formyl-C₁₋₄ alkyl, C₁₋₄ alkoxycarbonyl-C₁₋₄-alkyl, carbamoyl-C₁₋₄ alkyl, N—C₁₋₄ alkylcarbamoyl-C₁₋₄alkyl, N,N-di-[C₁₋₄ alkyl]carbamoyl-C₁₋₄alkyl, amino-C₁₋₄ alkyl, C₁₋₄ alkylamino-C₁₋₄ alkyl, di-[C₁₋₄ alkyl]amino-C₁₋₄ alkyl, phenyl-C₁₋₄ alkyl, 4-pyridon-1-yl-C₁₋₄ alkyl, pyrrolidin-1-yl-C₁₋₄ alkyl, imidazol-1-yl-C₁₋₄ alkyl, piperidino-C₁₋₄ alkyl, morpholino-C₁₋₄ alkyl, thiomorpholino-C₁₋₄alkyl, thiomorpholino-1-oxide-C₁₋₄alkyl, thiomorpholino-1,1-dioxide-C₁₋₄alkyl, piperazin-1-yl-C₁₋₄alkyl, 4-C₁₋₄ alkylpiperazin-1-yl-C₁₋₄ alkyl, hydroxy-C₂₋₄ alkoxy-C₁₋₄ alkyl, C₁₋₄ alkoxy-C₂₋₄ alkoxy-C₁₋₄ alkyl, hydroxy-C₂₋₄ alkylamino-C₁₋₄ alkyl, C₁₋₄ alkoxy-C₂₋₄ alkylamino-C₁₋₄ alkyl, C₁₋₄ alkylthio-C₁₋₄ alkyl, hydroxy-C₂₋₄ alkylthio-C₁₋₄ alkyl, C₁₋₄ alkoxy-C₂₋₄ alkylthio-C₁₋₄ alkyl, phenoxy-C₁₋₄ alkyl, anilino-C₁₋₄ alkyl, phenylthio-C₁₋₄ alkyl, cyano-C₁₋₄ alkyl, halogeno-C₂₋₄ alkoxy, hydroxy-C₂₋₄ alkoxy, C₂₋₄ alkanoyloxy-C₂₋₄ alkoxy, C₁₋₄ alkoxy-C₂₋₄ alkoxy, carboxy-C₁₋₄ alkoxy, formyl-C₁₋₄ alkoxy, C₁₋₄ alkoxycarbonyl-C₁₋₄ alkoxy, carbamoyl-C₁₋₄ alkoxy, N—C₁₋₄ alkylcarbamoyl-C₁₋₄ alkoxy, N,N-di-[C₁₋₄ alkyl]carbamoyl-C₁₋₄ alkoxy, amino-C₂₋₄ alkoxy, C₁₋₄ alkylamino-C₂₋₄ alkoxy, di-[C₁₋₄ alkyl]amino-C₂₋₄ alkoxy, di-[C₁₋₄ alkyl-C₂₋₄ alkoxyl]amino-C₂₋₄ alkoxy, C₂₋₄ alkanoyloxy, hydroxy-C₂₋₄ alkanoyloxy, C₁₋₄alkoxy-C₂₋₄ alkanoyloxy, phenyl-C₁₋₄ alkoxy, phenoxy-C₂₋₄ alkoxy, anilino-C₂₋₄ alkoxy, phenylthio-C₂₋₄ alkoxy, 4-pyridon-1-yl-C₂₋₄ alkoxy, piperidino-C₂₋₄ alkoxy, pyrrolidin-1-yl-C₂₋₄ alkoxy, imidazol-1-yl-C₂₋₄ alkoxy, morpholino-C₂₋₄ alkoxy, thiomorpholino-C₂₋₄ alkoxy, thiomorpholino-1-oxide-C₂₋₄ alkoxy, thiomorpholino-1,1-dioxide-C₂₋₄ alkoxy, piperazin-1-yl-C₂₋₄ alkoxy, 4-C₁₋₄ alkylpiperazin-1-yl-C₂₋₄ alkoxy, halogeno-C₂₋₄ alkylamino, hydroxy-C₂₋₄ alkylamino, C₂₋₄ alkanoyloxy-C₂₋₄ alkylamino, C₁₋₄ alkoxy-C₂₋₄ alkylamino, carboxy-C₁₋₄ alkylamino, C₁₋₄ alkoxycarbonyl-C₁₋₄ alkylamino, carbamoyl-C₁₋₄ alkylamino, N-C₁₋₄ alkylcarbamoyl-C₁₋₄ alkylamino, N,N-di-[C₁₋₄ alkyl]carbamoyl-C₁₋₄ alkylamino, amino-C₂₋₄ alkylamino, C₁₋₄ alkylamino-C₂₋₄ alkylamino, di-[C₁₋₄alkyl]amino-C₂₋₄ alkylamino, phenyl-C₁₋₄ alkylamino, phenoxy-C₂₋₄ alkylamino, anilino-C₂₋₄ alkylamino, 4-pyridon-1-yl-C₂₋₄ alkylamino, pyrrolidin-1-yl-C₂₋₄ alkylamino, imidazol-1-yl-C₂₋₄ alkylamino, piperidino-C₂₋₄ alkylamino, morpholino-C₂₋₄ alkylamino, thiomorpholino-C₂₋₄ alkylamino, thiomorpholino-1-oxide-C₂₋₄ alkylamino, thiomorpholino-1,1-dioxide-C₂₋₄ alkylamino, piperazin-1-yl-C₂₋₄ alkylamino, 4-(C₁₋₄ alkyl)piperazin-1-yl-C₂₋₄ alkylamino, phenylthio-C₂₋₄ alkylamino, C₂₋₄ alkanoylamino, C₁₋₄ alkoxycarbonylamino, C₁₋₄ alkylsulphonylamino, C₁₋₄ alkylsulphinylamino, benzamido, benzenesulphonamido, 3-phenylureido, 2-oxopyrrolidin-1-yl, 2,5-dioxopyrrolidin-1-yl, halogeno-C₂₋₄ alkanoylamino, hydroxy-C₂₋₄ alkanoylamino, hydroxy-C₂₋₄ alkanoyl-(C₁₋₄ alkyl)-amino, C₁₋₄ alkoxy-C₂₋₄ alkanoylamino, carboxy-C₂₋₄ alkanoylamino, C₁₋₄ alkoxycarbonyl-C₂₋₄ alkanoylamino, carbamoyl-C₂₋₄ alkanoylamino, N—C₁₋₄ alkylcarbamoyl-C₂₋₄ alkanoylamino, N,N-di-[C₁₋₄ alkyl]carbamoyl-C₂₋₄ alkanoylamino, amino-C₂₋₄ alkanoylamino, C₁₋₄ alkylamino-C₂₋₄ alkanoylamino or di-[C₁₋₄ alkyl]amino-C₂₋₄ alkanoylamino, and wherein said benzamido or benzenesulphonamido substituent or any anilino, phenoxy or phenyl group on a R¹ substituent may optionally bear one or two halo, C₁₋₄ alkyl or C₁₋₄ alkoxy substituents; and wherein any substituent containing a Het ring may optionally bear one or two halo, C₁₋₄ alkyl or C₁₋₄ alkoxy substituents on said ring; and wherein any substituent containing a Het ring may optionally bear one or two oxo or thioxo substituents on said ring;

or R¹ represents a group selected from M¹-M²-M³-M⁴, M¹-M⁵ or M¹-M²-M^{3′}-M⁶ wherein

M¹ represents a C₁₋₄ alkyl group, wherein optionally a CH₂ group is replaced by a CO group;

M² represents NR¹² or CR¹²R¹³, in which R¹² and R¹³ each independently represent H or C₁₋₄ alkyl;

M³ represents a C₁₋₄ alkyl group;

M³ represents a C₁₋₄ alkyl group or is absent;

M⁴ represents CN, NR¹²S(O)ₘR¹³, S(O)ₘNR¹⁴R¹⁵, CONR¹⁴R¹⁵, S(O)ₘR¹³ or CO₂R¹³, in which R¹², R¹³ and m are as hereinbefore defined and R¹⁴ and R¹⁵ each independently represent H or C₁₋₄ alkyl, or R¹⁴ and R¹⁵ together with the nitrogen atom to which they are attached represent a 5- or 6-membered ring optionally containing 1 or 2 additional heteroatoms selected from N, O or S(O)ₘ in which ring any nitrogen atom present may optionally be substituted with a C₁₋₄ alkyl group, and which ring may optionally bear one or two oxo or thioxo substituents;
M⁵ represents the group NR¹⁴R¹⁵, wherein R¹⁴ and R¹⁵ are as defined above, or M⁵ represents the group 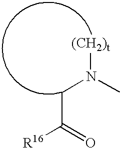
in which t represents 2 to 4 and R¹⁶ represents OH, OC₁₋₄ alkyl or NR¹⁴R¹⁵; and
M⁶ represents a C₃₋₆ cycloalkyl group, the group NR¹⁴R¹⁵, wherein R¹⁴ and R¹⁵ are as defined above, or a 5- or 6-membered Het ring system containing 1 to 4 heteroatoms selected from N, O or S;
and p is 0 to 3; or when p is 2 or 3, two adjacent R¹ groups together form an optionally substituted methylenedioxy or ethylenedioxy group;
R² is selected from the group comprising hydrogen, halogen, trifluoromethyl, C₁₋₄ alkyl and C₁₋₄ alkoxy;
U represents a 5 to 10-membered mono or bicyclic ring system in which one or more of the carbon atoms is optionally replaced by a heteroatom independently selected from N, O and S(O)ₘ, wherein m is 0, 1 or 2 and wherein the ring system is substituted by at least one independently selected R⁶ group and is optionally substituted by at least one independently selected R⁴ group;
each R⁴ is independently hydrogen, hydroxy, halogen, C₁₋₄ alkyl, C₁₋₄ alkoxy, C₁₋₄ alkylamino, di-[C₁₋₄ alkyl]amino, C₁₋₄ alkylthio, C₁₋₄ alkylsulphinyl, C₁₋₄ alkylsulphonyl, C₁₋₄ alkylcarbonyl, C₁₋₄ alkylcarbamoyl, di-[C₁₋₄ alkyl] carbamoyl, carbamyl, C₁₋₄ alkoxycarbonyl, cyano, nitro or trifluoromethyl;
each R⁶ is independently a group ZR⁷ wherein Z is joined to R⁷ through a (CH₂)ₚ group in which p is 0, 1 or 2 and Z represents a group V(CH₂), V(CF₂), (CH₂)V, (CF₂)V, V(CRR′), V(CHR) or V where R and R′ are each C₁₋₄ alkyl and in which V is a hydrocarbyl group containing 0, 1 or 2 carbon atoms, carbonyl, dicarbonyl, CH(OH), CH(CN), sulphonamide, amide, O, S(O)ₘ or NR^{b} where R^{b} is hydrogen or R^{b} is C₁₋₄ alkyl; and R⁷ is an optionally substituted C₃₋₆ cycloalkyl; or an optionally substituted 5, 6, 7, 8, 9 or 10-membered Cbc or Het moiety;
or R⁶ is a group ZR⁷ in which Z is NR^{b}, and NR^{b} and R⁷ together form an optionally substituted 5, 6, 7, 8, 9 or 10-membered carbocyclic or heterocyclic moiety.

Het groups comprise one or more rings which may be saturated, unsaturated, or aromatic and which may independently contain one or more heteroatoms in each ring.

Cbc groups comprise one or more rings which may be independently saturated, unsaturated, or aromatic and which contain only carbon and hydrogen.

Suitably the 5, 6, 7, 8, 9 or 10-membered Het moiety is selected from the group comprising: furan, dioxolane, thiophene, pyrrole, imidazole, pyrrolidine, pyran, pyridine, pyrimidine, morpholine, piperidine, oxazole, isoxazole, oxazoline, oxazolidine, thiazole, isothiazole, thiadiazole, benzofuran, indole, isoindole, quinazoline, quinoline, isoquinoline and ketal.

Suitably the 5, 6, 7, 8, 9 or 10-membered Cbc moiety is selected from the group comprising: phenyl, benzyl, indene, naphthalene, tetralin, decalin, cyclopentyl, cyclopentenyl, cyclohexyl, cyclohexenyl and cycloheptyl.

In an embodiment, R¹ is as defined above with the exception of wherein any substituent containing a Het ring bears one or two oxo or thioxo substituents on said ring; and R¹⁴ and R¹⁵ are as defined above with the exception of wherein they together with the nitrogen atom to which they are attached represent a 5- or 6-membered ring and said ring bears one or two oxo or thioxo substituents; save that R¹ may represent 4-pyridon-1-yl, 4-pyridon-1-yl-C₁₋₄ alkyl, 4-pyridon-1-yl-C₂₋₄ alkoxy, 4-pyridon-1-yl-C₂₋₄ alkylamino, 2-oxopyrrolidin-1-yl or 2,5-dioxopyrrolidin-1-yl.

In a further embodiment, R¹ is selected from the group comprising amino, hydrogen, halogen, hydroxy, formyl, carboxy, cyano, nitro, C₁₋₈ alkyl, C₁₋₈ alkoxy, C₁₋₈ alkylthio, C₁₋₈ alkylsulphinyl, C₁₋₈ alkylsulphonyl, C₁₋₄ alkylamino, C₁₋₄ dialkylamino, dioxolanyl, benzyloxy or hydroxy-C₁₋₄ alkanoyl-(C₁₋₄ alkylamino.

In a preferred embodiment, R¹ is selected from the group comprising amino, C₁₋₄ alkylamino, diC₁₋₄ alkylamino, especially diC₁₋₄ alkylamino, most especially dimethylamino or methylethylamino.

In a further embodiment, R¹ is selected from M¹-M²-M³-M⁴, M¹-M⁵ or M¹-M²-M^{3′}-M⁶ as defined above; and p=1.

In a further embodiment, the group M²-M³-M⁴ represents an α-, β- or γ-amino carboxylic, sulphinic or sulphonic acid or a C₁₋₄ alkyl ester, an amide or a C₁₋₄ alkyl- or di-(C₁₋₄ alkyl)-amide thereof.

Preferably M¹ represents CH₂, CO, CH₂CH₂ or CH₂CO, more preferably CH₂.

Preferably M² represents NR¹² in which R¹² is as defined above; more preferably R¹² represents H or methyl.

Preferably M³ represents CH₂, CH₂CH₂ or propyl.

Preferably M^{3′} represents CH₂, ethyl, propyl, isopropyl or is absent.

Preferably M⁴ represents SOR¹³, SO₂R¹³, NR¹²SO₂R¹³, CO₂R¹³ or CONR¹⁴R¹⁵ in which R¹² and R¹³ are defined above and R¹⁴ and R¹⁵ each independently represent H or C₁₋₄ alkyl; more preferably R¹², R¹³, R¹⁴ and R¹⁵ each independently represent H or methyl.

Preferably M⁵ represents a group NR¹⁴R¹⁵ in which R¹⁴ and R¹⁵ together with the nitrogen atom to which they are attached represent a 6-membered ring optionally containing an additional heteroatom selected from N or O, in which ring any nitrogen atom present may optionally be substituted with a C₁₋₄ alkyl group, preferably a methyl group; or M⁵ represents a group 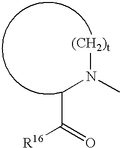
in which t represents 2 or 3 and R¹⁶ represents OH, NH₂, N(C₁₋₄ alkyl)₂ or OC₁₋₄ alkyl; more preferably R¹⁶ represents NH₂ or N(CH₃)₂.

M⁵ also preferably represents a group NR¹⁴R¹⁵ in which R¹⁴ and R¹⁵ each independently represent hydrogen or C₁₋₄ alkyl, more preferably hydrogen, methyl, ethyl or isopropyl.

Preferably M⁶ represents a group NR¹⁴R¹⁵ in which R¹⁴ and R¹⁵ each independently represent C₁₋₄ alkyl, more preferably methyl, or R¹⁴ and R¹⁵ together with the nitrogen atom to which they are attached represent a 5- or 6-membered ring optionally containing an additional heteroatom selected from N or O, in which ring any nitrogen atom present may optionally be substituted with a C₁₋₄ alkyl group, preferably a methyl group; or M⁶ represents a 5- or 6-membered Het ring system containing 1 or 2 heteroatoms selected from N or O.

In a further preferred embodiment, M²-M³-M⁴ represents an α-amino carboxylic acid or a methyl ester or amide thereof.

In a further preferred embodiment, M²-M³-M⁴ represents an α-, β- or γ-amino sulphinic or sulphonic acid, more preferably a β- or γ-amino sulphinic or sulphonic acid, most preferably a β-aminosulphonic acid, or a methyl ester thereof.

In an especially preferred embodiment, M²-M³-M⁴ represents a methylsulphonylethylamino, methylsulphinylethylamino, methylsulphonylpropylamino, methylsulphinylpropylamino, methylsulphonamidoethylamino, sarcosinamide, glycine, glycinamide, glycine methyl ester or acetylaminoethylamino group.

In a further especially preferred embodiment, M⁵ represents a piperazinyl, methylpiperazinyl, piperidinyl, prolinamido or N,N-dimethylprolinamido group.

In a further especially preferred embodiment, M⁵ represents an isopropylamino or N-morpholinyl group.

In a further especially preferred embodiment, M¹-M⁵ represents an isopropylacetamido or N-morpholinoacetamido group.

In a further especially preferred embodiment, M²-M^{3′}-M⁶ represents a pyridylamino, cyclopropylamino, N-(piperidin-4-yl)-N-methylamino, N,N-dimethylaminoprop-2-ylamino, N-(2-dimethylaminoethyl)-N-ethylamino or tetrahydrofuranomethylamino group, preferably a pyridylamino group.

In a further embodiment, each R¹ is independently selected from the group comprising amino, hydrogen, halogen, hydroxy, formyl, carboxy, cyano, nitro, C₁₋₈ alkyl, C₁₋₈ alkoxy, C₁₋₈ alkylthio, C₁₋₈ alkylsulphinyl, C₁₋₈ alkylsulphonyl, C₁₋₄ alkylamino, C₁₋₄ dialkylamino, benzyloxy, hydroxy-C₁₋₄ alkyl, hydroxy-C₁₋₄ alkanoyl-(C₁₋₄ alkyl)-amino.

In an embodiment, R² is hydrogen, C₁₋₄ alkyl, C₁₋₄ alkoxy or halogen, preferably methyl or hydrogen, more preferably hydrogen.

In a further embodiment, R⁴ is hydrogen, hydroxy, halogen, C₁₋₄ alkyl, C₁₋₄ alkoxy, di-[C₁₋₄ alkyl]amino, nitro or trifluoromethyl, preferably hydrogen, halogen or methyl, more preferably hydrogen.

In a preferred embodiment, R⁷ is an optionally substituted phenyl, dioxolanyl, thienyl, cyclohexyl or pyridyl group.

In a further embodiment, Z is absent or represents oxygen, CH₂, NR^{b}, NR^{b}(CH₂), (CH₂)NR^{b}, CH(CH₃), O(CH₂), (CH)CN, O(CF₂), (CH₂)O, (CF₂)O, S(CH₂), S(O)ₘ, carbonyl or dicarbonyl, wherein R^{b} is hydrogen or C₁₋₄ alkyl.

In a preferred embodiment, Z is oxygen, dicarbonyl, OCH₂, CH₂(CN), S(O)ₘ or NR^{b}, wherein R^{b} is hydrogen or C₁₋₄ alkyl.

In a further preferred embodiment, R⁶ is benzyl, halo-, dihalo- and trihalobenzyl, α-methylbenzyl, phenyl, halo-, dihalo- and trihalophenyl, pyridyl, pyridylmethyl, pyridyloxy, pyridylmethoxy, thienylmethoxy, dioxolanylmethoxy, cyclohexylmethoxy, phenoxy, halo-, dihalo- and trihalophenoxy, phenylthio, benzyloxy, halo-, dihalo- and trihalobenzyloxy, C₁₋₄ alkoxybenzyloxy, phenyloxalyl or benzenesulphonyl, more preferably benzyl, fluorobenzyl, benzyloxy, fluorobenzyloxy, pyridylmethyl, phenyl, benzenesulphonyl, phenoxy or fluorophenoxy.

In a further embodiment, R⁶ is in the para position with respect to the aniline N.

When the group Z is absent, R⁶═R⁷.

One or both of the rings comprising the mono or bicyclic ring system U may be aromatic or non-aromatic. The R⁴ and R⁶ groups may be bound to the ring system by either a carbon atom or a heteroatom of the ring system. The ring system itself may be bound to the bridging group by a carbon atom or a heteroatom. The R⁴ and R⁶ groups may be bound to either ring when U represents a bicyclic ring system, but these groups are preferably bound to the ring, which is not bound to the bridging group Y in such a case.

Examples of suitable mono or bicyclic groups U include: isoindenyl, indenyl, indanyl, naphthyl, 1,2-dihydronaphthyl or 1,2,3,4-tetrahydronaphthyl, pyrrolyl, pyridinyl, pyridazinyl, pyrimidinyl, pyrazinyl, furanyl, 2H-pyranyl, thiophenyl, 1H-azepinyl, oxepinyl, thiepinyl, azocinyl, 2H-oxocinyl, thieno[2,3-b]furanyl, thianaphthenyl, indolyl, indolinyl, isoindolyl, isoindolinyl, indolizinyl, 1H-benzimidazolyl, 2,3-dihydro-1H-benzimidazolyl, 1H-indazolyl, 2,3-dihydro-1H-indazolyl, benzoxazolyl, 2,3-dihydrobenzoxazolyl, benzo[c]isoxazolyl, benzo[d]isoxazolyl, 2,3-dihydrobenzo[d]isoxazolyl, benzothiazoyl, 2,3-dihydrobenzothiazolyl, benzo[c]isothiazolyl, benzo[d]isothiazolyl, 2,3-dihydrobenzo[d]isothiazolyl, 1H-benzotriazolyl, benzo[c]furanyl, benzo[c][1,2,3]thiadiazolyl, benzo[d][1,2,3]oxadiazolyl, benzo[d][1,2,3]thiadiazolyl, quinolyl, 1,2-dihydroquinolinyl, 1,2,3,4-tetrahydroquinolinyl, isoquinolyl 1,2,3,4-tetrahydroisoquinolinyl, cinnolinyl, quinazolinyl, quinoxalinyl, phthalazinyl, 4H-1,4-benzoxazinyl, 2,3-dihydro-4H-1,4-benzoxazinyl, 4H-1,4-benzothiazinyl or 2,3-dihydro-4H-1,4-benzothiazinyl.

Suitably U represents an indolyl, isoindolyl, indolinyl, isoindolinyl, 1H-indazolyl, 2,3-dihydro-1H-indazolyl, 1H-benzimidazolyl, 2,3-dihydro-1H-benzimidazolyl or 1H-benzotriazolyl group.

In an embodiment, the optional substitutents for the Cbc or Het moiety, which may be present at any available position of said moiety, are selected from the group comprising:

(CH₂)_{q}S(O)ₘ—C₁₋₄alkyl, (CH₂)_{q}S(O)ₘ—C cycloalkyl, (CH₂)_{q}SO₂NR⁸R⁹, (CH₂)_{q}NR⁸R⁹, (CH₂)_{q}CO₂R⁸, (CH₂)_{q}OR⁸, (CH₂)_{q}CONR⁸R⁹, (CH₂)_{q}NR⁸COR⁹, (CH₂)_{q}COR⁸, (CH₂)_{q}R⁸, NR⁸SO₂R⁹ and S(O)ₘR⁸,

wherein q is an integer from 0 to 4 inclusive; m is 0, 1 or 2;

R⁸ and R⁹ are independently selected from the group comprising hydrogen, C₁₋₄ alkyl, C₃₋₆ cycloalkyl, aryl, a 5- or 6-membered saturated or unsaturated Het ring which may be the same or different and which contains one or more heteroatoms which are selected from N, O or S(O)ₘ, with the proviso that the Het ring does not contain two adjacent O or S(O)ₘ atoms.

In a further embodiment, the optional substitutents for the Cbc or Het moiety are selected from the group comprising morpholine, piperazine, piperidine, pyrrolidine, tetrahydrofuran, dioxolane, oxothiolane and oxides thereof, dithiolane and oxides thereof, dioxane, pyridine, pyrimidine, pyrazine, pyridazine, furan, thiofuran, pyrrole, triazine, imidazole, triazole, tetrazole, pyrazole, oxazole, oxadiazole and thiadiazole.

Other optional substituents for the Cbc or Het moiety and also for other optionally substituted groups include, but are not limited to, hydroxy, halogen, trifluoromethyl, trifluoromethoxy, nitro, amino, cyano, C₁₋₄ alkoxy, C₁₋₄ alkylthio, C₁₋₄ alkyl carbonyl, carboxylate and C₁₋₄ alkoxy carboxyl.

In a further preferred embodiment, there is provided a compound of formula (I″) or a salt, solvate, or physiologically functional derivative thereof, wherein R^{a} is hydrogen or C₁₋₄ alkyl; R¹ group is selected from hydrogen, halo, C₁₋₄ alkyl, carboxy, formyl, hydroxy-C₁₋₄ alkyl, 1,3-dioxolan-2-yl, benzyloxy, amino, C₁₋₄ alkylamino, di(C₁₋₄ alkyl)amino, hydroxy-C₁₋₄ alkanoyl(C₁₋₄ alkyl)amino, C₁₋₄ alkylamino-C₁₋₄ alkyl, di(C₁₋₄ alkyl)amino-C₁₋₄ alkyl, methylsulphonylethylaminomethyl, methylsulphonylethylamino-carbonyl, methylsulphinylethylamino-methyl, methylsulphinylethylamino-carbonyl, methylsulphonylpropylamino-methyl, methylsulphinylpropylamino-methyl, methylsulphonylpropyamino-carbonyl, methylsulphinylpropylamino-carbonyl, methylsulphonylethyl-(methylamino)-methyl, methylsulphonylethyl-(methylamino)-carbonyl, methylsulphinylethyl-(methylamino)-methyl, methylsulphinylethyl-(methylamino)-carbonyl, methylsulphonylpropyl-(methylamino)-methyl, methylsulphinylpropyl-(methylamino)-methyl, methylsulphonylpropyl-(methylamino)-carbonyl, methylsulphinylpropyl-(methylamino)-carbonyl, methylsulphonamidoethylamino-methyl, methylsulphonamidopropylamino-methyl, sarcosinamidomethyl, glycinylmethyl, glycinamidomethyl, glycinylmethyl methyl ester, acetylaminoethylaminomethyl, piperazinylmethyl, methylpiperazinylmethyl, piperidinylmethyl, N-(prolinamido)methyl, (N,N-dimethyl-prolinamido)methyl, pyridylaminomethyl, cyclopropylaminomethyl, N-(piperidin-4-yl)-N-methylaminomethyl, N,N-dimethylaminoprop-2-ylaminomethyl, N-(2-dimethylaminoethyl)-N-ethylaminomethyl, isopropylacetamido, N-morpholinylacetamido or tetrahydrofuranomethylaminomethyl; R² represents hydrogen; R⁴ represents hydrogen or methyl; U represents indolyl, benzimidazolyl or indazolyl, more preferably indazolyl; and R⁶ represents phenyl, benzyl, α-methylbenzyl, fluorobenzyl, benzenesulphonyl, phenoxy, fluorophenoxy, benzyloxy or fluorobenzyloxy.

In a further especially preferred embodiment, there is provided a compound of formula (I″) or a salt, solvate, or physiologically functional derivative thereof wherein R^{a} is hydrogen or C₁₋₄ alkyl; R¹ group is selected from hydrogen, halo, benzyloxy, amino, C₁₋₄ alkylamino, di(C₁₋₄ alkyl)amino or hydroxy-C₁₋₄ alkanoyl(C₁₋₄ alkyl)amino, more preferably dimethylamino; R² represents hydrogen; R⁴ represents hydrogen or methyl; U represents indazolyl, indolyl or benzimidazolyl, more preferably indazolyl; and R⁶ represents benzyl, fluorobenzyl, pyridylmethyl or benzenesulphonyl.

A preferred species of a compound of Formula (1″) is:

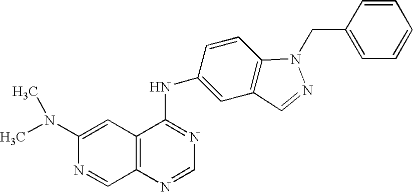

The compounds of Formula (I″) may be prepared according to the procedures of U.S. Pat. No. 6,174,889 and according to the appropriate Examples recited below.

As recited above the method and treatment combination of the present invention also includes at least one of a PI3K and an Akt inhibitor. Generally any AkT inhibitor, that is, any pharmaceutical agent having specific Akt inhibitor activity may be utilized in the present invention. Such Akt inhibitors are described, for instance, in WO2002083064, WO2002083138, WO2002083140, WO2002083139, WO2002083675, WO2003010281, WO200198290, WO03014090, WO200248114, WO2003013517, WO200230423, WO2002057259, WO200222610, WO2003011854, WO2003084473, and WO2003011855, which patent applications are herein incorporated by reference to the extent of their disclosure of Akt inhibitor compounds and methods of making and using the same.

In one embodiment of the present invention, the Akt inhibitor is a compound of the Formula IV: 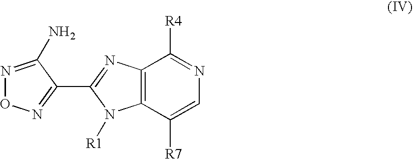
wherein: R¹ is selected from: hydrogen, alkyl, alkyl substituted with one or more substituents selected from the group consisting of: hydroxy, alkoxy, amino, N-acylamino, cyclopropyl and halogen, cycloalkyl, cycloalkyl substituted with one or more substituents selected from the group consisting of: hydroxy, alkoxy, amino, N-acylamino and halogen, cycloalkyl containing from 1 to 3 heteroatoms, cycloalkyl containing from 1 to 3 heteroatoms substituted with one or more substituents selected from the group consisting of: hydroxy, alkoxy, amino, N-acylamino and halogen, C₁-C₁₂aryl and C₁-C₁₂aryl substituted with one or more substituents selected from the group consisting of: hydroxy, alkoxy, amino, N-acylamino and halogen;
R⁴ is selected from hydrogen, halogen, alkyl, substituted alkyl, cycloalkyl, cycloalkyl containing from 1 to 3 heteroatoms, and a cyclic or polycyclic aromatic ring containing from 3 to 16 carbon atoms and optionally containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR², —C(O)NR⁵R⁶, —S(O)₂NR⁵R⁶, —S(O)ₙR² and protected —OH,
where n is 0-2,
R² is hydrogen, alkyl, cycloalkyl, C₁-C₁₂aryl, substituted alkyl, substituted cycloalkyl and substituted C₁-C₁₂aryl, and
R⁵ and R⁶ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH,
or R⁵ and R⁶ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2; and
R⁷ is selected from hydrogen, —C(O)NR⁹R¹⁰, —(CH₂)ₙNR⁹R¹⁰, —SO₂NR⁹R¹⁰ and —(CH₂)ₙOR⁸,
where n is 0-2;
R⁸ is alkyl, cycloalkyl, cycloalkyl containing from 1 to 3 heteroatoms, piperidyl and pyrrolidinyl, each of which is optionally substituted with one or more substituents selected from the group consisting of: alkoxy, acyloxy, aryloxy, amino, N-acylamino, oxo, hydroxy, —C(O)OR², —S(O)ₙR², —C(O)NR²R³, —S(O)₂NR²R³, nitro, cyano, cycloalkyl, substituted cycloalkyl, halogen, aryl, substituted aryl and protected —OH, where R² and R³ are independently hydrogen, alkyl, cycloalkyl, C₁-C₁₂aryl, substituted alkyl, substituted cycloalkyl and substituted C₁-C₁₂aryl, and n is 0-2,
R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —C(O)OR², —S(O)ₙR², —C(O)NR²R³, —S(O)₂NR²R³, —NR²R³, nitro, cyano, cycloalkyl, substituted cycloalkyl, halogen, aryl, substituted aryl and protected —OH,
or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino,
where R² and R³ are independently hydrogen, alkyl, cycloalkyl, C₁-C₁₂aryl, substituted alkyl, substituted cycloalkyl and substituted C₁-C₁₂aryl, and n is 0-2;
or pharmaceutically acceptable salts and solvates thereof.

Included among the presently invented compounds of Formula (IV) are those in which:

R¹ is selected from: alkyl, alkyl substituted with one or more substituents selected from the group consisting of: hydroxy, alkoxy, amino, N-acylamino, cyclopropyl and halogen, cycloalkyl containing from 1 to 3 heteroatoms and C₁-C₁₂aryl;

R⁴ is selected from hydrogen, halogen, alkyl, substituted alkyl, cycloalkyl, cycloalkyl containing from 1 to 3 heteroatoms, C₁-C₁₂aryl and C₁-C₁₂aryl substituted with one or more substituents selected from the group consisting of: alkyl, substituted alkyl, aryloxy, hydroxy, alkoxy, acyloxy, amino, N-acylamino, nitro, cyano and halogen; and

R⁷ is selected from hydrogen, —C(O)NR⁹R¹⁰ and —(CH₂)ₙOR⁸, where n is 0-2;

R⁸ is alkyl, piperidine, imidazolidine, piperidyl and pyrrolidinyl, each of which is optionally substituted with one or more substituents selected from the group consisting of: alkoxy, acyloxy, aryloxy, amino, N-acylamino, hydroxy, nitro, cyano, cycloalkyl, halogen and C₁-C₁₂aryl,

R⁹ and R¹⁰ are independently hydrogen, cycloalkyl, cycloalkyl containing from 1 to 3 heteroatoms, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, methylamino, dimethylamino, hydroxyalkyl, —NR²R³, nitro, cyano, cycloalkyl, halogen, aryl and substituted aryl,

or R⁹ and R¹⁰ taken together with the nitrogen to which they are attached represent a 5 to 6 member saturated ring containing up to one other heteroatom selected from oxygen and nitrogen, where the ring is optionally substituted with one or more substituents selected from amino, methylamino and dimethylamino, where R² and R³ are independently hydrogen, alkyl, cycloalkyl, C₁-C₁₂aryl, substituted alkyl, substituted cycloalkyl and substituted C₁-C₁₂aryl;

and pharmaceutically acceptable salts, hydrates, solvates and esters thereof.

A group of preferred compounds of the formula (IV) is selected from the group:
4-{1-ethyl-4-phenyl-7-[(3-piperidinylmethyl)oxy]-1H-imidazo[4,5-c]pyridin-2-yl}-1,2,5-oxadiazol-3-amine trifluoroacetate;
4-{4-(3-chlorophenyl)-1-ethyl-7-[(4-piperidinylmethyl)oxy]-1H-imidazo-[4,5-c]pyridin-2-yl}-1,2,5-oxadiazol-3-amine trifluoroacetate;
4-[7-[(4-aminobutyl)oxy]-4-(3-chlorophenyl)-1-ethyl-1H-imidazo-[4,5-c]pyridin-2-yl]-1,2,5-oxadiazol-3-amine trifluoroacetate;
4-{7-[(3-aminopropyl)oxy]-1-ethyl-4-phenyl-1H-imidazo[4,5-c]pyridin-2-yl}-1,2,5-oxadiazol-3-amine trifluoroacetate;
2-(4-amino-1,2,5-oxadiazol-3-yl)-4-(3-chlorophenyl)-1-(cyclopropylmethyl)-N-{2-[(phenylmethyl)amino]ethyl}-1H-imidazo[4,5-c]pyridine-7-carboxamide; and
4-[1-ethyl-7-(piperidin-4-yloxy)-1H-imidazo[4,5-c]pyridin-2-yl]-furazan-3-ylamine;
   and salts, solvates, and physiologically functional derivatives thereof.

For compounds of Formula (IV):

The term “aryl” is as defined above.

The term “C₁-C₁₂aryl” as used in formula IV, unless otherwise defined, is meant phenyl, naphthalene, 3,4-methylenedioxyphenyl, pyridine, biphenyl, quinoline, pyrimidine, quinazoline, thiophene, furan, pyrrole, pyrazole, imidazole benzothiophene and tetrazole.

The term “substituted” as used in formula IV, unless otherwise defined, is meant that the subject chemical moiety has one or more substituents selected from the group consisting of: —CO₂R²⁰, aryl, —C(O)NHS(O)₂R²⁰, —NHS(O)₂R²⁰, hydroxyalkyl, alkoxy, —C(O)NR²¹R²², acyloxy, alkyl, amino, methylamino, dimethylamino, N-acylamino, hydroxy, —(CH₂)_{g}C(O)OR²³, —S(O)ₙR²³, nitro, tetrazole, cyano, oxo, halogen, trifluoromethyl and protected —OH, where g is 0-6, R²³ is hydrogen or alkyl, R²⁰ is selected form hydrogen, C₁-C₄alkyl, aryl and trifluoromethyl, and R²¹ and R²² are independently selected form hydrogen, C₁-C₄alkyl, aryl and trifluoromethyl, and n is 0-2.

The term “alkoxy” is as defined above including —OCH₃ and —OC(CH₃)₂CH₃.

The term “cycloalkyl” is as defined above herein.

Examples of cycloalkyl and substituted cycloalkyl substituents as used in formula IV herein include: cyclohexyl, 4-hydroxy-cyclohexyl, 2-ethylcyclohexyl, propyl 4-methoxycyclohexyl, 4-methoxycyclohexyl, 4-carboxycyclohexyl, cyclopropyl and cyclopentyl.

The term “acyloxy” is defined as described above. Examples of acyloxy substituents as used herein for formula (IV) include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

Term “aryloxy” is as described above optionally substituted with one or more substituents selected from the group consisting of: alkyl, hydroxyalkyl, alkoxy, trifuloromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used in formula (IV) include: phenoxy, 4-fluorophenyloxy and biphenyloxy.

The term “heteroatom” as used in formula (IV) is meant oxygen, nitrogen or sulfur.

The term “alkyl” is as defined above. Examples of alkyl substituents as used in formula (IV) include: —CH₃, —CH₂—CH₃, —CH₂—CH₂-CH₃, —CH(CH₃)₂, —C(CH₃)₃, —(CH₂)₃—CH₃, —CH₂—CH(CH₃)₂, —CH(CH₃)—CH₂—CH₃, —CH═CH₂, and —C≡C—CH₃.

The compounds of Formula (IV) may be prepared similarly to Examples 8-13 below.

Another Akt inhibitor useful in the present invention is 4-[1-Ethyl-7-(piperidin-4-ylmethoxy)-1H-imidazo[4,5-c]pyridin-2-yl]-furazan-3-ylamine.

The at least one PI3K inhibitor may be any suitable PI3K inhibitor, that is any pharmaceutical agent having specific PI3K inhibitor activity may be utilized in the present invention.

One PI3K inhibitor compound that may be usefully employed in the present invention is wortmannin. Wortmannin is a fungal metabolite obtained from Penicillium fumiculosum. Wortmannin (CAS [19545-26-7] is a off-white to pale yellow solid having a molecular weight of 428.4. The compound may be purchased commercially, for instance from A.G. Scientific, Inc.).

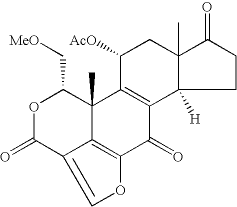

Wortmannin

Another PI3K inhibitor compound that may be usefully employed in the present invention is LY294002. LY294002 (CAS[15447-36-6] is a selective PI3K inhibitor which has a molecular weight of 307.3 and may be purchased commercially, for instance from Cayman Chemical.

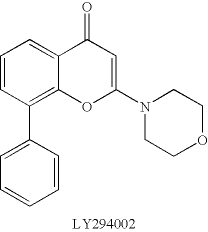

The erb family inhibitor, e.g., dual EGFR/erbB-2 inhibitor and the PI3K and/or Akt inhibitor, may be employed in combination in accordance with the invention by administration concomitantly in (1) a unitary pharmaceutical composition including both compounds or (2) separate pharmaceutical compositions each including one of the compounds. Alternatively, the combination may be administered separately in a sequential manner wherein, for example, the PI3K or Akt inhibitor or dual EGFR/erbB-2 inhibitor is administered first and the other second. Such sequential administration may be close in time or remote in time.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts derived from a nitrogen on a substituent in a compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium and valerate. Other salts, which are not pharmaceutically acceptable, may be useful in the preparation of compounds of this invention and these form a further aspect of the invention.

While it is possible that, for use in therapy, therapeutically effective amounts of a dual EGFR/erbB2, PI3K or Akt inhibitor, as well as salts, solvates and physiological functional derivatives thereof, may be administered as the raw chemical, it is possible to present the active ingredient as a pharmaceutical composition. Accordingly, the invention further provides pharmaceutical compositions, which include therapeutically effective amounts of a dual EGFR/erbB2 and/or PI3K or Akt inhibitor and salts, solvates and physiological functional derivatives thereof, and one or more pharmaceutically acceptable carriers, diluents, or excipients. The compounds of the present invention and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a dual EGFR/erbB2 and/or a PI3K or Akt inhibitor or salts, solvates and physiological functional derivatives thereof, with one or more pharmaceutically acceptable carriers, diluents or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of an EGFR/erbB2 and/or PI3K or Akt inhibitor, depending on the condition being treated, the route of administration and the age, weight and condition of the patient, or pharmaceutical formulations may be presented in unit dose forms containing a predetermined amount of active ingredient per unit dose. Preferred unit dosage formulations are those containing a daily dose or sub-dose, as herein above recited, or an appropriate fraction thereof, of an active ingredient. Furthermore, such pharmaceutical formulations may be prepared by any of the methods well known in the pharmacy art.

The dual EGFR/erbB-2 inhibitors and PI3K or Akt inhibitors may be administered by any appropriate route. Suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination. It will also be appreciated that each of the agents administered may be administered by the same or different routes and that the erbB-2 and PI3K or Akt inhibitors may be compounded together in a pharmaceutical composition/formulation.

The method of the present invention may also be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged. Anti-neoplastic therapies are described for instance in International Application No. PCT US 02/01130, filed Jan. 14, 2002, which application is incorporated by reference to the extent that it discloses anti-neoplastic therapies. Combination therapies according to the present invention thus include the administration of at least one erbB-2 inhibitor and at least one PI3K and/or Akt inhibitor as well as optional use of other therapeutic agents including other anti-neoplastic agents. Such combination of agents may be administered together or separately and, when administered separately this may occur simultaneously or sequentially in any order, both close and remote in time. The amounts of the erbB2, PI3K, and Akt inhibitors and the other pharmaceutically active agent(s) and the relative timings of administration will be selected in order to achieve the desired combined therapeutic effect.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also to granulating, the powder mixture can be run through the tablet machine and the result is imperfectly formed slugs broken into granules. The granules can be lubricated be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to prevent sticking to the tablet forming dies by means of the addition of stearic acid, a stearate salt, talc or mineral oil. The lubricated mixture is then compressed into tablets. The compounds of the present invention can also be combined with free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxyl ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The agents for use according to the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Agents for use according to the present invention may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists that may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavoring agents.

Also, contemplated in the present invention is a pharmaceutical combination including at least one erb family inhibitor, such as a dual erbB-2/EGFR inhibitor and at least one PI3K and/or Akt inhibitor. In another embodiment, the pharmaceutical combination includes an erbB-2 inhibitor, a PI3K inhibitor and/or Akt inhibitor, and optionally at least one additional anti-neoplastic agent. The erb inhibitors, PI3K and Akt inhibitors, and additional anti-neoplastic therapy are as described above.

As indicated, therapeutically effective amounts of the specific erb family inhibitor and PI3K and/or Akt inhibitor are administered to a mammal. Typically, the therapeutically effective amount of one of the administered agents of the present invention will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the erb family and PI3K and/or Akt inhibitors will be given in the range of 0.1 to 100 mg/kg body weight of recipient (mammal) per day and more usually in the range of 1 to 10 mg/kg body weight per day.

As indicated, the method of cancer treatment of the present invention, is directed to any susceptible cancer. Typically, the cancer is any cancer which is susceptible to inhibition of EGFR, erbB-2, Akt and/or PI3K. Examples of cancers that are suitable for treatment by the method and treatment combination of the present invention include, but are limited to, head and neck, breast, lung, colon, ovary, and prostate cancers.

The following examples are intended for illustration only and are not intended to limit the scope of the invention in any way.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| N (Normal) | Kg (kilogram) |
| i.v. (intravenous); | Hz (Hertz); |
| MHz (megahertz); | mol (moles); |
| mmol (millimoles); | RT (room temperature); |
| min (minutes); | h (hours); |
| mp (melting point); | TLC (thin layer chromatography); |
| Tᵣ (retention time); | RP (reverse phase); |
| DCM (dichloromethane); | DCE (dichloroethane); |
| DMF (N,N-dimethylformamide); | HOAc (acetic acid); |
| TMSE (2-(trimethylsilyl)ethyl); | TMS (trimethylsilyl); |
| TIPS (triisopropylsilyl); | TBS (t-butyldimethylsilyl); |
| HPLC (high pressure liquid | DMSO (dimethylsulfoxide); |
| chromatography); | DME (1,2-dimethoxyethane); |
| THF (tetrahydrofuran); | |
| EtOAc (ethyl acetate); | |
| EDTA | ethylenediaminetetraacetic |
| | acid |
| FBS | fetal bovine serum |
| IMDM | Iscove's Modified |
| | Dulbecco's medium |
| PBS | phosphate buffered saline |
| RPMI | Roswell Park Memorial |
| | Institute |
| RIPA buffer | * |
| RT | room temperature |

*150 mM NaCl, 50 mM Tris-HCl, ph 7.5, 0.25% (w/v)-deoxycholate, 1% NP-40, 5 mM sodium orthovanadate, 2 mM sodium fluoride, and a protease inhibitor cocktail.

Unless otherwise indicated, all temperatures are expressed in ° C. (degrees Centigrade). All reactions conducted under an inert atmosphere at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, 8 units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APIiii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck). Optical rotations were obtained using a Perkin Elmer Model 241 Polarimeter. Melting points were determined using a Mel-Temp II apparatus and are uncorrected.

Examples 1-7 recite the preparation of specific erbB-2/EGFR inhibitors useful in the present invention.

### Example 1

### Monohydrate ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (monohydrate ditosylate salt of compound of formula (III))

### 1(a) Preparation of N-{3-Chloro-4[(3-fluorobenzyl)oxy]phenyl}-6-[5-({[2-(methane sulphonyl)ethyl]amino}methyl)-2-furyl]-4-quinazolinamine (free base of compound of formula (III))

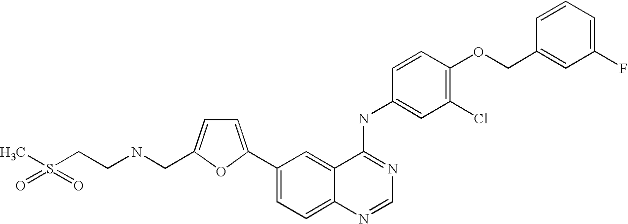

The title compound was prepared according to Procedure D of International Applications WO 02/02552: p. 16, line 19 to p. 17, line 3 and WO 99/35146: p. 56, lines 20-32 and Example 29 p. 100, lines 18-29, from 5-(4-{3-chloro-4-(3-fluorobenzyloxy)-anilino}-6-quinazolinyl)-furan-2-carbaldehyde (0.6 equiv) and 2-methanesulphonyl-ethylamine (1 equiv). ¹H NMR 400 MHz (DMSO-d6) 9.60 (bs, 1H); 9.32 (bs, 1H); 8.82 (bs, 1H); 8.34 (d, 1H); 8.0 (s, 1H); 7.88 (d, 1H); 7.74 (d, 1H); 7.45 (m, H); 7.34-7.23 (m, 4H); 7.17 (m, 1H); 6.83 (d, 1H); 5.27 (s, 2H); 4.42 (s, 2H); 3.59 (m, 2H); 3.40 (m, 2H, obscured by waterpeak); 3.12 (s, 3H); MS m/z 581 (M+H⁺).

### 1(b) Preparation of monohydrate ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (monohydrate ditosylate salt of compound of formula (III)

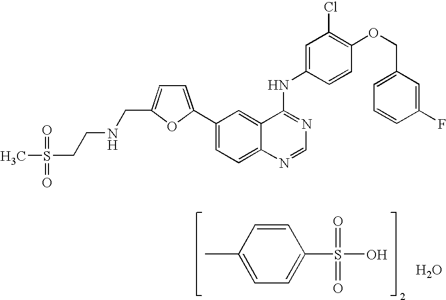

### Stage 1: Preparation of N-{3-chloro-4-[(3-fluorobenzyl)oxy]phenyl}-6-iodo-4-quinazolnamine

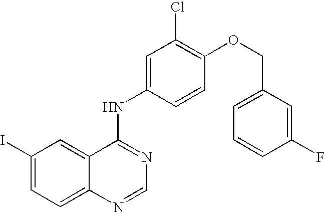

4-Chloro-6-iodoquinazoline (1 wt) was added to a solution of fluorobenzyloxyaniline (0.894 wt, 1.03 equiv) in N-methylpyrrolidinone (8.26 wt, 8 vol) at ca 20° C., and after the initial exotherm had subsided, the resulting solution was stirred at 20°-25° C. for at least 30 minutes. The dark solution was treated with triethylamine (0.58 vol, 1.2 equiv) and the mixture was stirred for 20-30 minutes. Isopropanol (2.5 vol) was added and the mixture was heated to ca 50° C. Water (up to 3 vol) was added slowly to the vessel over 10-15 minutes, while keeping the temperature at ca 50° C. Once crystallisation had commenced the addition was stopped and the resulting slurry was aged for 30-45 minutes at ca 50° C. Any residual water (from the 3 vol) was added, then further water (5 vol) was added to the vessel over ca 30 minutes while maintaining the temperature at ca 50° C. The resulting slurry was cooled to ca 20° C. over ca 30 minutes and aged at ca 20° C. for at least 30 minutes. The solid was collected by filtration and washed sequentially with water (2×5 vol), then isopropanol (5 vol). The product was dried in vacuo at ca 60° C. to give the title compound as a cream crystalline solid.

### Stage 2: Preparation of 5-(4-[3-chloro-4-(3-fluorobenzyloxy)-anilino]-6-quinazolinyl)-furan-2-carbaldehyde 4-methylbenzenesulfonate

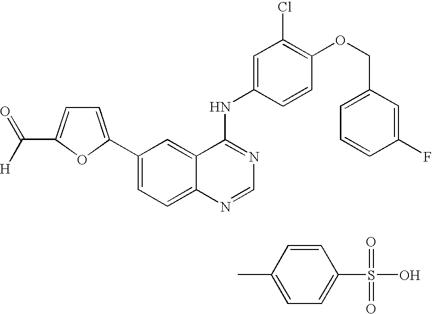

A mixture of N-{3-chloro-4-[(3-fluorobenzyl)oxy]phenyl}-6-iodo-4-quinazolinamine (1 wt), boronic acid (0.37 wt, 1.35 equiv), and 10% palladium on charcoal (0.028 wt, 50% water wet) was slurried in IMS (15 vol). The resultant suspension was stirred for 5 minutes, treated with di-isopropylethylamine (0.39 vol, 1.15 equiv) and then heated to ca 70° C. for ca 3 hours when the reaction was complete (determined by HPLC analysis). The mixture was diluted with tetrahydrofuran (THF, 15 vol) and then filtered (hot—through GFA filter paper) to remove catalyst. The vessel was rinsed with IMS (2 vol).

A solution of p-toluenesulfonic acid monohydrate (1.54 wt, 4.1 equiv) in water (3 vol) was added over 5-10 minutes to the filtered solution maintained at 65° C. After crystallisation the suspension was stirred at 60°-65° C. for 1 hour, cooled to ca 25° C. over 1 hour and stirred at this temperature for a further 2 hours. The solid was collected by filtration, washed with IMS (3 vol) then dried in vacuo at ca 50° C. to give the tile compound as a yellow-orange crystalline solid.

### Stage 3: Preparation of anhydrous ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (anhydrous ditosylate salt of compound of formula (III))

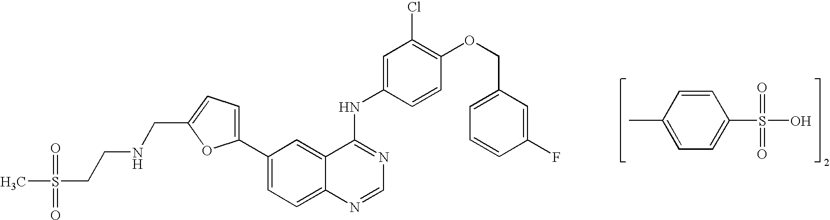

5-(4-[3-chloro-4-(3-fluorobenzyloxy)-anilino]-6-quinazolinyl)-furan-2-carbaldehyde 4-methylbenzenesulfonate (1 wt) and 2-(methylsulfonyl)ethylamine hydrochloride (0.4 wt, 1.6 equiv) were suspended in THF (10 vol). Sequentially, acetic acid (0.35 vol, 4 equiv) and di-isopropylethylamine (1.08 vol, 4 equiv) were added. The resulting solution was stirred at 30°-35° C. for ca 1 hour then cooled to ca 23° C. Sodium triacetoxyborohydride (0.66 wt, 2 equiv) was then added as a continual charge over approximately 15 minutes (some effervescence is seen at this point). The resulting mixture was stirred at ca 22° C. for ca 2 hours then sampled for HPLC analysis. The reaction was quenched by addition of 5M aqueous sodium hydroxide (5 vol) and stirred for ca 30 minutes (some effervescence is seen at the start of the caustic addition).

The aqueous phase was then separated, extracted with THF (2 vol) and the combined THF extracts were then washed with 10% w/v aqueous sodium chloride solution (4 vol). A solution of p-toluenesulfonic acid monohydrate (pTSA, 1.77 wt, 6 equiv) in THF (7 vol)¹ was prepared and warmed to ca 55° C. The THF solution of N-{3-Chloro-4-[(3-fluorobenzyl)oxy]phenyl}-6-[5-({[2-(methanesulphonyl)ethyl]amino}methyl)-2-furyl]-4-quinazolinamine was added to the pTSA solution over at least 30 minutes, maintaining the batch temperature at ca 55°±3° C.². The resulting suspension was stirred at ca 55° C. for 2 hours, cooled to 20°-25° C. over ca 60 minutes and aged at this temperature for ca 30 minutes. The solid was collected by filtration, washed with THF (2×2 vol) and dried in vacuo at ca 40° C. to give the desired compound as a pale yellow crystalline solid.

### Stage 4: Preparation of monohydrate ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (monohydrate ditosylate salt of compound of formula (III))

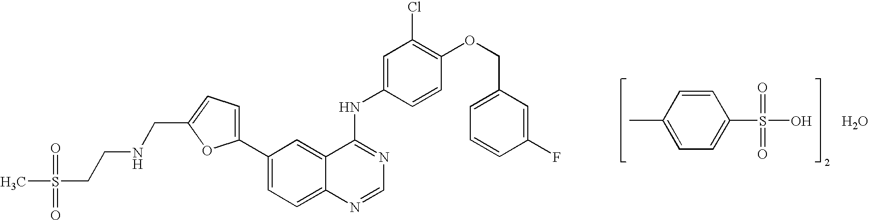

A suspension of the anhydrous ditosylate salt of N-{3-Chloro-4-[(3-fluorobenzyl)oxy]phenyl}-6-[5-({[2-(methane sulphonyl)ethyl]amino}methyl)-2-furyl]-4-quinazolinamine (1 wt), in tetrahydrofuran (THF, 14 vol) and water (6 vol) was heated to ca 55°-60° C. for 30 minutes to give a solution which was clarified by filtration and the lines washed into the crystallisation vessel with THF/Water (7:3, 2 vol). The resultant solution was heated to reflux and tetrahydrofuran (9 vol, 95% w/w azeotrope with water) was distilled off at atmospheric pressure.

The solution was seeded with N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate (0.002 wt). Once the crystallisation was established water (6 vol) was added while maintaining the reaction temperature above 55° C. The mixture was cooled to 5°-15° C. over ca 2 hours. The solid was collected by filtration, washed with tetrahydrofuran/water (3:7 ratio, 2×2 vol) and dried in vacuo at 45° C. to give N{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate as a bright yellow crystalline solid.

### Example 2

### N-{3-chloro-4-[(3-fluorobenzyl)oxy]phenyl}-6-(5-{[2-(methylsulfonyl)ethoxy]methyl}-2-furyl)-4-quinazolinamine

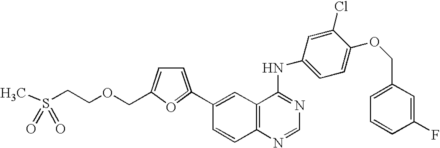

Prepared according to Procedure O of WO 01/04111 (referred to above) utilizing 3-[5-(4-{3-chloro-4-[(3-fluorobenzyl)oxy]anilino}-6-quinazolinyl)-2-furyl]-2-methen alcohol (66.8 mg, 0.141 mmol), methyl vinyl sulfone (0.015 mL, 0.169 mmol) and sodium hydride (60% in mineral oil, 0.7 mg, 0.017 mmol) in DMF (3 mL) to provide the title compound (51 mg) after purification by chromatography. ¹H NMR 400 MHz (DMSO-d6) 9.95 (1H, s), 8.74 (1H, s), 8.50 (1H, s), 8.11 (1H, d, J=8.8 Hz), 7.96 (1H, s), 7.76-7.68 (2H, m), 7.41 (1H, m), 7.29-7.22 (3H, m), 7.11 (1H, m), 7.06 (1H, d, J=2.8 Hz), 6.65 (1H, d, J=2.8 Hz), 5.21 (2H, s), 4.55 (2H, s), 3.81 (2H, t), 3.37 (2H, t), 2.94 (3H, s). LC/MS m/z 582 (M+H⁺).

### Example 3

### 2-{{[5-(4-{3-chloro-4-[(3-fluorobenzyl)oxy]anilino}-6-quinazolinyl-2-furyl]methyl}[2-(methylsulfonyl)ethyl]amino}acetonitrile

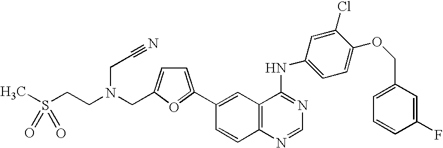

(4-(3-Fluorobenzyloxy)-3-chlorophenyl)-(6-(2-((2-methanesulphonyl-ethylamino)-ethyl)-furan-2-yl)-quinazolin-4-yl)-amine (116 mg, 0.2 mmol), chloroacetonitrile (0.014 mL, 0.22 mmol) and diisopropyl ethyl amine (0.07 mL, 0.2 mmol) were mixed, as outlined in Procedure P of WO 01/04111, to provide the title compound (110 mg). ¹H NMR 400 MHz (DMSO-d6) 9.84 (1H, s), 8.69 (1H, s), 8.50 (1H, s), 8.10 (1H, d, J=8.8 Hz), 7.96 (1H, d, J=2.4 Hz), 7.76 (1H, d, J=8.8 Hz), 7.68 (1H, m), 7.42 (1H, m), 7.29-7.22 (3H, m), 7.13 (1H, m), 7.03 (1H, d, J=3.6 Hz), 6.59 (1H, d, J=3.6 Hz), 5.22 (2H, s), 3.84 (2H, s), 3.81 (2H, s), 3.37 (2H, t), 2.98 (3H, s), 2.96 (2H, t). LC/MS m/z 620 (M+H⁺).

### Example 4

### (4-(3-Fluorobenzyloxy)-3-chlorophenyl)-(6-(2-((2-iso-propyl-sulphonyl-ethylamino)-methyl)-furan-2-yl)-quinazolin-4-yl)-amine

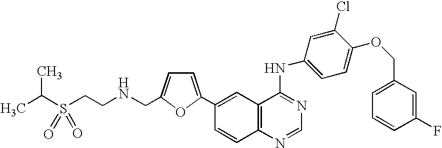

The title compound and its hydrochloride salt are prepared according to Procedure D of WO 01/047111 (page 97), utilizing 5-{-4-[4-(3-fluoro-benzyloxy)-3-chloroanilino]-6-quinazolinyl}-2-furaldehyde (0.317 mmol, 0.15 g), Isopropylsulfonylethyl amine hydrochloride salt (0.475 mmol, 0.105 g) in the presence of Et₃N (0.95 mmol, 0.13 mL) and NaBH₄ (1.1 mmol, 0.041 g) in THF/MeOH. ¹H NMR (DMSO-d6) 11.74 (bs, 1H); 9.90 (bs, 2H); 9.63 (s, 1H); 8.91 (s, 1H); 8.42 (d, 1H); 8.04 (m, 1H); 7.95 (d, 1H); 7.81 (d, 1H); 7.47 (m, 1H); 7.37-7.28 (m, 4H); 7.18 (m, 1H); 6.83 (m, 1H); 5.29 (s, 2H); 4.45 (s, 2H); 3.72-3.39 (m, 5H); 1.26 (d, 6H). Electrospray MS 609.

### Example 5

### N4-(1-Benzyl-1H-indazol-5-yl)-N6,N6-dimethyl-pyrido[3,4-d]pyrimidine-4,6-diamine

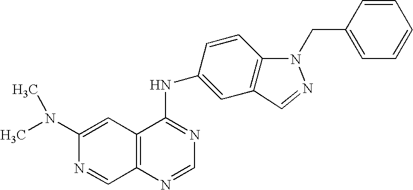

A stirred solution of (1-benzyl-1H-indazol-5-yl)-(6-chloro-pyrido[3,4-d]pyrimidin-4-yl)-amine (0.5 g) in 33% aqueous dimethylamine (5 ml) was heated at 130° C. in a reacti-vial for 17 hr. The cooled mixture was dissolved in chloroform, absorbed onto silica and chromatographed to give the title compound (Procedure C: Col 20, lines 10-16 of U.S. Pat. No. 6,174,889) as a yellow solid; δH [²H₆]-DMSO 9.00 (1H,s), 8.51 (1H,s), 8.09 (2H,d), 7.55 (1H,dd), 7.25 (7H,m), 6.39 (1H,m), 5.60 (2H,s) 3.20 (6H,s); m/z (M+1)⁺396.

### Example 6

### Preparation of (4-(3-Fluoro-benzyloxy)-3-bromophenyl)-(6-(5-((2-methanesulphonyl-ethylamino)-methyl)-furan-2-yl)quinazolin-4-yl)-amine ditosylate. (The ditosylate Salt of the Compound of Formula (III″)

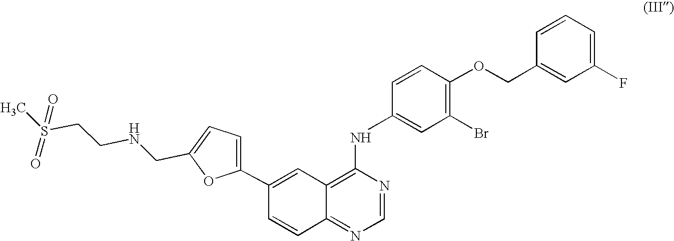

The HCl salt of 5-(4-[3-bromo-4-(3-fluorobenzyloxy)-anilino]-6-quinazolinyl)-furan-2-carbaldehyde (prepared according to Procedure C, page 56 of WO 99/35146) was converted to the tosylate salt according to the procedure of Example 1, Stage 2. The resultant furan 2-carbaldehyde tosylate product was used to prepare the (4-(3-Fluoro-benzyloxy)-3-bromophenyl)-(6-(5-((2-methanesulphonyl-ethylamino)-methyl)-furan-2-yl)quinazolin-4-yl)-amine ditosylate according to the procedure of Example 1, stage 3.

### Example 7

### Preparation of (4-(3-Fluoro-benzyloxy)-3-chlorophenyl)-(6-(2-((2-methanesulphonyl-ethylamino)-methyl)-thiazol-4-yl)quinazolin-4-yl)-amine ditosylate (ditosylate Salt of the Compound of Formula III′)

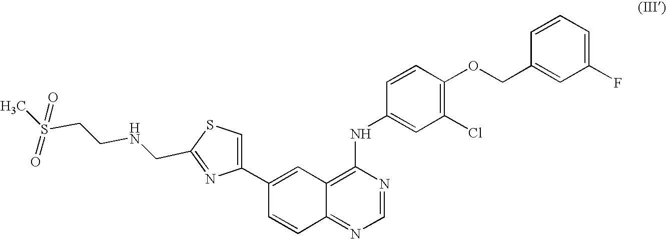

The HCL salt of (4-(3-Fluoro-benzyloxy)-3-chlorophenyl)-(6-(2-((2-methanesulphonyl-ethylamino)-methyl)-thiazol-4-yl)quinazolin-4-yl)-amine was prepared according to Procedure F, pages 57-59 of WO 99/35146 and then converted to the (4-(3-Fluoro-benzyloxy)-3-chlorophenyl)-(6-(2-((2-methanesulphonyl-ethylamino)-methyl)-thiazol-4-yl)quinazolin-4-yl)-amine ditosylate salt according to the procedures of Example 1.

Examples 8-9 recite the preparation of specific Akt inhibitors useful in the present invention.

### Example 8

### Preparation of 2-(4-amino-1,2,5-oxadiazol-3-yl)-4-(3-chlorophenyl)-1-(cyclopropylmethyl)-N-{2-[(phenylmethyl)amino]ethyl}-1H-imidazo[4,5-c]pyridine-7-carboxamide, trifluoroacetate salt

### a) Cyclopropylmethyl-(3-nitropyridin-4-yl)amine

4-Ethoxy-3-nitropyridine, hydrochloride (14.5 g, 70.8 mmol) in ethyl acetate was washed twice with 1N NaHCO₃. The organic layer was dried over MgSO4, filtered and the solvent evaporated under reduced pressure to give 11.8 g of a light tan solid. The free-amine (11.8 g, 69.9 mmol) and cyclopropanemethylamine (5.00 g, 70.3 mmol) in EtOH were heated at 80° C. in a sealed tube for 12 h. After allowing to warm to RT, the solvent was removed under reduced pressure to give a yellow oil. Flash chromatography (silica gel, hexanes then hexanes/Et₂O (1:1:1) then Et₂O/CH₂Cl₂ (1:1) then CH₂Cl₂) gave 13.1 g of the desired material. MS (ES) m/z 194.2 [M+H]⁺.

### b) (3-Bromo-5-nitropyridin-4-yl)cyclopropylmethylamine

To the compound of Example 8(a) (13.1 g, 68.0 mmol) and NaOAc (25.1 g, 305.5 mmol) in glacial acetic acid (20 mL) was added bromine (15.6 g, 97.6 mmol). The reaction was maintained at 100° C. for 20 h. After cooling to room temperature, the mixture was diluted with CH₂Cl₂ and filtered. The solvent was removed from the filtrate under reduced pressure and the residue purified by flash chromatography (silica gel, 0% to 20% EtOAc/hexanes) to give 9.81 g of the desired product as a yellow oil. MS (ES) m/z 272.2 [M+H]⁺.

### c) 5-Bromo-2-chloro-N⁴-cyclopropylmethylpyridine-3,4-diamine

The compound of Example 8(b) (3.11 g, 11.43 mmol) was dissolved into ethanol (25 mL) and cooled to 0° C. Concentrated HCl (25 mL) was added while maintaining the reaction at 0° C. After 15 min., tin (II) chloride (6.55 g, 34.5 mmol) was added. After 3 h at 0° C., the reaction mixture was poured into a solution of NaOH (24 g, 600 mmol) in ice water (75 mL). The mixture was extracted with EtOAc and the combined organic extracts were dried over MgSO₄. The solvent was removed under reduced pressure to give 3.05 g of the desired material. This was used without further purification. MS (ES) m/z 276.0 [M+H]⁺.

### d) [7-Bromo-4-chloro-1-(cyclopropylmethyl)-1H-imidazo[4,5-c]pyridin-2-yl]acetonitrile

The compound of Example 8(c) (2.60 g, 9.40 mmol) in ethyl cyanoacetate (10.6 g, 93.8 mmol) was heated to 190° C. for 3 h. The reaction was allowed to cool to RT. Flash chromatography (silica gel, 50% Et₂O/CHCl₃) gave 1.62 g of the desired material. MS(ES) m/z 325.0 [M+H]⁺.

### e) (7-Bromo-4-chloro-1-cyclopropylmethyl-1H-imidazo[4,5-c]pyridin-2-yl)hydroxyimino-acetonitrile

To the compound of Example 8(d) (1.32 g, 4.65 mmol) in MeOH (30 mL) and 2N HCl (15 mL) was added sodium nitrite (0.59 g, 8.55 mmol). After stirring at RT for 1 h, the precipitate was collected by filtration and dried under vacuum to give 1.35 g of the desired material as a yellow powder. This was used without further purification. MS (ES) m/z 354.0 [M+H]⁺.

### f) 2-(7-Bromo-4-chloro-1-cyclopropylmethyl-1H-imidazo[4,5-c]pyridin-2-yl)-N-hydroxy-2-hydroxyimino acetamidine

To the compound of Example 8(e) (1.35 g, 3.80 mmol) and Et₃N (1.46 g, 14.4 mmol) in THF (20 mL) was added hydroxylamine (0.70 mL, 10.6 mmol). The reaction was heated at 90° C. for 1 h. After allowing to cool to RT, the reaction was diluted with EtOAc and washed with H₂O and brine. The organic extract was dried over MgSO₄ and the solvent was removed under reduced pressure to give 1.56 g of the desired material as a yellow oil. This was used without further purification. MS (ES) m/z 387.0 [M+H]⁺.

### g) 4-(7-Bromo-4-chloro-1-cyclopropylmethyl-1H-imidazo[4,5-c]pyridin-2-yl)furazan-3-ylamine

The compound of Example 8(f) (1.57 g, 3.80 mmol) and Et3N (2.18 g, 21.5 mmol) in 1,4-dioxane was heated at 150° C. in a sealed tube for 1 h. After allowing to cool to RT, the crude reaction mixture purified by flash chromatography (silica gel, 0% to 20% EtOAc/hexanes) to give 0.90 g of the desired product as a cream colored solid. MS (ES) m/z 368.8 [M+H]⁺.

### h) [4-(7-Bromo-4-chloro-1-cyclopropylmethyl-1H-imidazo[4,5-c]pyridin-2-yl)furazan-3-yl]di-tert-butoxycarbonylamine

To the compound of Example 8(g) (0.90 g, 2.43 mmol) in CHCl₃ (20 mL) was added di-tert-butyldicarbonate (1.12 g, 5.14 mmol) and dimethylaminopyridine (67.7 mg, 0.55 mmol). The reaction was heated to reflux for 1 h. After allowing to cool to RT, the solvent was removed under reduced pressure. Trituration from hot MeOH gave 1.06 g of the desired material as a white powder. MS (ES) m/z 569.2 [M+H]⁺.

### i) 4-Chloro-1-(cyclopropylmethyl)-2-[4-(({[(1,1-dimethylethyl)oxy]carbonyl}amino)-1,2,5-oxadiazol-3-yl]-1H-imidazo[4,5-c]pyridine-7-carboxylic acid

A solution of the compound of Example 8(h) (0.84 g, 1.48 mmol) in dry THF (25 mL) was degassed and then cooled to −78° C. n-Butyllithium (1.50 mL of a 2.50 M solution in hexanes, 3.75 mmol) was added to the cooled solution. After 5 minutes, CO₂ gas was bubbled into the reaction for 1 h while continuing to maintain the reaction at −78° C. The reaction was allowed to reach ambient temperature and diluted with EtOAc. The organic layer was washed with 1N NaOH and dried over MgSO₄. The solvent was removed under reduced pressure. Purification by preparative reverse phase HPLC (Phenomenex® Synergi MaxRP 80A column, gradient 10% AcCN/H₂O to 80% AcCN/H₂O+0.1% TFA) gave 0.14 g of the desired material as a grey solid. MS(ES) m/z 435.4 [M+H]⁺.

### j) 4-(3-Chlorophenyl)-1-(cyclopropylmethyl)-2-[4-({[(1,1-dimethylethyl)oxy]carbonyl}amino)-1,2,5-oxadiazol-3-yl]-1H-imidazo[4,5-c]pyridine-7-carboxylic acid

To a solution of the compound of Example 8(i) (100 mg, 0.23 mmol) and 3-chlorophenylboronic acid (47.5 mg, 0.30 mmol) in EtOH (10 mL) and toluene (10 mL) was added 2M Na₂CO₃ (0.70 mL, 1.40 mmol). The mixture was degassed and (Ph₃P)₄Pd (48.1 mg.; 0.04 mmol) was added. The reaction was heated to reflux for 6 h. After allowing to cool to RT, the reaction mixture was filtered and the filtrate was concentrated. Flash chromatography (silica gel, 10% to 25% EtOH/CHCl₃) gave 135 mg of the desired material as a white solid. MS (ES) m/z 511.4 [M+H]⁺.

### k) 1,1-Dimethylethyl (4-{4-(3-chlorophenyl)-1-(cyclopropylmethyl)-7-[({2-[(phenylmethyl)amino]ethyl}amino)carbonyl]-1H-imidazo[4,5-c]pyridin-2-yl}-1,2,5-oxadiazol-3-yl)carbamate

To the compound of Example 80) (37.2 mg, 0.073 mmol) in CH₂Cl₂ and DMF was added N-benzylethylenediamine (20 mg, 0.13 mmol), 1-hydroxy-7-azabenzotriazole (17.5 mg, 0.13 mmol), 1-(3-dimethylaminopropyl)-3-ethylcarbodiimide, hydrochloride (27.8 mg, 0.15 mmol) and triethylamine (43.7 mg, 0.43 mmol). After 6 d at RT, the solvent was removed under reduced pressure. Flash chromatography (silica gel, 5% MeOH/CHCl₃) gave 27.4 mg of the desired material as a tan oil. MS (ES) m/z 643.4 [M+H]⁺.

### l) 2-(4-Amino-1,2,5-oxadiazol-3-yl)-4-(3-chlorophenyl)-1-(cyclopropylmethyl)-N-{2-[(phenylmethyl)amino]ethyl}-1H-imidazo[4,5-c]pyridine-7-carboxamide, trifluoroacetate salt

The compound of Example 8(k) (27.4 mg) was dissolved in CH₂Cl₂ (10 mL) and trifluoroacetic acid (10 mL). After 1 h at RT, the solvent was removed under reduced pressure. Trituration with Et₂O gave 13.8 mg of the title compound as a white powder. MS (ES) m/z 543.4 [M+H]⁺.

### Example 9

### Preparation of 4-[1-Ethyl-7-(piperidin-4-yloxy)-1H-imidazo[4,5-c]pyridin-2-yl]-furazan-3-ylamine

### a) Ethyl (3-nitropyridin-4-yl)amine

A solution consisting of 4-methoxy-3-nitropyridine (15.0 g, 97.3 mmol) with ethyl amine (46.5 mL of 70% aqueous solution, 584 mmol) in ethanol (30 mL) was stirred at 85° C. in a sealed flask for 2 h. Removal of all volatiles in vacuo afforded the title compound (16.2 g, 99%). MS: (M+H)⁺=m/z 168.

### b) Ethyl (3-bromo-5-nitropyridin-4-yl)amine

A mixture consisting of ethyl (3-nitropyridin-4-yl)amine (11.76 g, 70 mmol) in acetic acid (140 ml) with sodium acetate (28.7 g, 350 mmol) and bromine (13.44 g, 84 mmol) was stirred in a sealed flask at 100° C. for 18 h. Most of the solvent was removed in vacuo and the residue partitioned between CH₂Cl₂ and water and the aqueous layer basified with NaHCO₃. The organic extract was washed with water then brine, dried (Na₂SO₄) and all volatiles removed in vacuo. The residue was chromatographed on silica gel eluted with ethyl acetate:hexane (2:8) to afford the title compound (10.4 g, 60%). MS: (M+H)=m/z 246.

### c) 5-Bromo-N⁴-ethyl-pyridine-3,4-diamine

A mixture of ethyl (3-bromo-5-nitropyridin-4-yl)amine (7.0 g, 28.4 mmol) in acetic acid (100 mL) with iron powder (<50 micron, 9.51 g, 170 mmol) was stirred at 75° C. for 1 h. The reaction mixture was cooled then diluted with EtOAc:CH₂Cl₂ (1:4) and filtered through celite. The filtrate was concentrated in vacuo then chromatographed on silica gel eluted with ethyl acetate:methanol (96:4) to afford the title compound (5.68 g, 92.7%). MS: (M+H)⁺=m/z 216.

### c) (7-Bromo-1-ethyl-1H-imidazo[4,5-c]pyridin-2-yl)-acetonitrile

A solution of 5-Bromo-N⁴-ethyl-pyridine-3,4-diamine (5.68 g, 26.3 mmol) in ethyl cyanoacetate (5.6 mL, 52.6 mmol) was stirred at 190° C. for 1 h. The product crystallized on cooling and addition of EtOAc (50 mL). The solid was collected, washed with EtOAc then dried to afford the title compound (4.15 g, 59%). MS: (M+H)⁺=m/z 265.

### e) 4-(7-Bromo-1-ethyl-1H-imidazo[4,5-c]pyridin-2-yl)-[1,2,5]oxadiazolidin-3-ylamine

To a solution of (7-bromo-1-ethyl-1H-imidazo[4,5-c]pyridin-2-yl)-acetonitrile (3.2 g, 12.1 mmol) in methanol (40 mL) was added in portions sodium nitrite (1.67 g, 24.2 mmol). The resulting mixture was stirred 2 h then adjusted to pH 12 with 50% aqueous NaOH. To this was added 50% aqueous NH₂OH (33 ml) and the mixture was stirred at 90° C. for 2 h. The solid which formed on cooling was collected by filtration to afford the title compound (2.50 g, 67%). MS: (M+H)⁺=m/z 309.

### f) [4-(7-Bromo-1-ethyl-1H-imidazo[4,5-c]pyridin-2-yl)-furazan-3-yl]-carbamic acid tert-butyl ester

A solution consisting of 4-(7-bromo-1-ethyl-1H-imidazo[4,5-c]pyridin-2-yl)-[1,2,5]oxadiazolidin-3-ylamine (2.14 g, 6.95 mmol) in methylene chloride (10 mL) and pyridine (20 mL) with di-t-butyl dicarbonate (2.27 g, 10.43 mmol) and DMAP (0.85 g, 6.95 mmol) was stirred at 90° C. in a sealed tube for 2.5 h. Additional di-t-butyl dicarbonate (2.27 g, 10.43 mmol) was added and stirring at 90° C. continued for 18 h. The product mixture was partitioned between EtOAc and water, the layers separated and the organic extract washed with water then brine, dried (Na₂SO₄) and all volatiles removed in vacuo. The residue was chromatographed on silica 20% EtOAc in hexane to afford the title compound as an off-white solid 1.60 g, 58.4%) MS: (M+H)⁺=m/z 409.

### g) [4-(1-Ethyl-7-hydroxy-1H-imidazo[4,5-c]pyridin-2-yl)-furazan-3-yl]-carbamic acid tert-butyl ester

To a solution of [4-(7-bromo-1-ethyl-1H-imidazo[4,5-c]pyridin-2-yl)-furazan-3-yl]-carbamic acid tert-butyl ester (205 mg, 0.5 mmol) in THF (4 mL) stirred at −78° C. under N₂ was added n-BuLi (0.5 ml of 2.5 M solution in hexane, 1.25 mmol). This was stirred at −78° C. for 20 min then trimethyl borate (168 uL, 1.5 mmol) with THF (1 mL) was added. Stirring was continued for 1.5 h while the reaction mixture was allowed to warm to room temperature. To the resulting mixture was added a solution consisting of 30% H₂O₂ (1.1 mL) in 3N NaOH (0.35 mL) and stirring continued at room temperature for 30 min. The reaction mixture was diluted with EtOAc then washed with 1N NaOH (3×). The combined aqueous extract was acidified with 6N HCl and the product extracted into EtOAc. The organic extract was dried (Na₂SO₄) and all volatiles removed in vacuo to afford the product as an orange solid (144 mg, 83%). MS: (M+H)⁺=m/z 347.

### h) 4-[2-(4-tert-Butoxycarbonylamino-furazan-3-yl)-1-ethyl-1H-imidazo[4,5-c]pyridin-7-yloxy]-piperidine-1-carboxylic acid tert-butyl ester

To a stirred mixture of triphenyl phosphine polystyrene (2.4 g, 1.2 mmol/g, 2.88 mmol) in CH₂Cl₂ (25 mL) was added 4-hydroxypiperidine-1-carboxylic acid tert-butyl ester (1.15 g, 5.76 mmol) followed by diethyl azodicarboxylate (0.45 mL, 2.88 mmol). After 10 min at room temperature the mixture was cooled to 0° C. and a solution of [4-(1-ethyl-7-hydroxy-1H-imidazo[4,5-c]pyridin-2-yl)-furazan-3-yl]-carbamic acid tert-butyl ester (200 mg, 0.58 mmol) in CH₂Cl₂ (15 mL) was added. This was stirred 1.5 h at 0° C. then filtered. the resin was washed with CH₂Cl₂ and the combined organic extract washed with 1 N NaOH soln then water, dried (Na₂SO₄) and all volatiles removed. The residue was purified by preparative HPLC (eluted with CH₃CN/H₂O/0.1% TFA) to afford the title compound as an off white solid (131 mg, 43%). MS: (M+H)⁺=m/z 530.

### i) 4-[1-Ethyl-7-(piperidin-4-yloxy)-1H-imidazo[4,5-c]pyridin-2-yl]-furazan-3-ylamine

A solution of 4-[2-(4-tert-butoxycarbonylamino-furazan-3-yl)-1-ethyl-1H-imidazo[4,5-c]pyridin-7-yloxy]-piperidine-1-carboxylic acid tert-butyl ester (130 mg, 0.25 mmol) in CH₂Cl₂ (1.5 mL) with TFA (0.75 mL) was stirred at room temperature for 40 min. Removal of all volatiles followed by purification by preparative HPLC (eluted with CH₃CN/H₂O) afforded the title compound (80 mg, 97%). MS: (M+H)⁺=m/z 330.

### Example 10

### Preparation of 4-{1-ethyl-4-phenyl-7-[(3-piperidinylmethyl)oxy]-1H-imidazo[4,5-c]pyridin-2-yl}-1,2,5-oxadiazol-3-amine trifluoroacetate

### a) Ethyl(3-nitropyridin-4-yl)amine

A solution consisting of 4-ethoxy-3-nitropyridine (15.0 g, 97.3 mmol) and EtNH₂ (46.5 mL, 70% aq. solution, 584 mmol) in EtOH (30 mL) was stirred at 85° C. in a pressure vessel for 2 h. Removal of all volatiles in vacuo afforded the title compound (16.2 g, 99%). MS (ES+) m/z 168(M+H)⁺.

### b) Ethyl (3-bromo-5-nitropyridin-4-yl)amine

A mixture of ethyl (3-nitropyridin-4-yl)amine (11.8 g, 70.0 mmol), acetic acid (140 mL), sodium acetate (28.7 g, 0.35 mol) and bromine (13.4 g, 84.0 mmol) was stirred in a pressure vessel at 100° C. for 18 h. The solvent was removed in vacuo and the residue partitioned between CH₂Cl₂ and water. The aqueous layer was made basic (pH˜8) with NaHCO₃ and further extracted with CH₂Cl₂. The combined organic extracts were washed with water, brine and dried (Na₂SO₄). The solvent was removed in vacuo. and the residue subjected to flash chromatography (20% EtOAc/hexanes, silica gel) to give 10.4 g (60%) of the desired compound. MS (ES+) m/z 246(M+H)⁺.

### c) 5-Bromo-2-chloro-N⁴-ethyl-pyridine-3,4-diamine

To a solution of ethyl (3-bromo-5-nitropyridin-4-yl)amine (22.0 g, 89.4 mmol) in conc HCl (250 mL) was added in portions tin (II) chloride dihydrate (60.5 g, 270 mmol). The mixture was stirred at RT for 1 h and then poured onto ice (300 g). EtOAc (500 mL) was added and the mixture made basic (pH˜10) with solid NaOH. The aqueous layer was extracted with EtOAC and the combined organic layers were washed with water, brine and dried (Na₂SO₄). The solvent was removed in vacuo. and the residue subjected to flash chromatography (25% EtOAc/hexanes, silica gel) to give 17.8 g (80%) of the desired compound. MS (ES+) m/z 250(M+H)⁺.

### d) N-(5-Bromo-2-chloro-4-ethylamino-pyridin-3-yl)-cyanoacetamide

To a solution of 5-bromo-2-chloro-N⁴-ethyl-pyridine-3,4-diamine (17.7 g, 70.9 mmol) in DMF (100 mL) at 0° C. was added cyanoacetic acid (9.06 g, 106 mmol), N-methyl morpholine (39 mL, 350 mmol) and EDCl (20.3 g, 106 mmol). The cooling bath was removed and stirring continued 3 h. The reaction was diluted with EtOAc (300 mL) and washed with water and brine. The solvent was removed in vacuo to give a solid. Recrystallization from EtOAc/hexanes afforded the desired compound (22.5 g). MS (ES+) m/z 317(M+H)⁺.

### e) (7-Bromo-4-chloro-1-ethyl-1H-imidazo[4,5c]pyridin-2-yl)-acetonitrile

A solution of N-(5-bromo-2-chloro-4-ethylamino-pyridin-3-yl)-cyanoacetamide (35.6 g, 112 mmol) in acetic acid (300 mL) was stirred at 90° C. for 1 h. The solvent was removed in vacuo to give the desired compound (29.5 g). This was used without further purification. MS (ES+) m/z 299(M+H)⁺.

### f) (7-Bromo-4-chloro-1-ethyl-1H-imidazo[4,5-c]pyridin-2-yl)-hydroxyimino-acetonitrile

To a mixture of (7-bromo-4-chloro-1-ethyl-1H-imidazo[4,5-c]pyridin-2-yl)-acetonitrile (29.5 g, 98 mmol) in 2 N HCl (400 mL) at RT was added portion wise over 20 min sodium nitrite (14.0 g, 203 mmol). After stirring for an additional 30 min the resulting precipitate isolated by filtration, washed with water and dried to afford the desired compound (32 g). This was used without further purification. MS (ES+) m/z 328(M+H)⁺.

### g) 4-(7-Bromo-4-chloro-1-ethyl-1H-imidazo[4,5-c]pyridin-2-yl)-1,2,5-oxadiazol-3-amine

A solution of the compound of Example 10(f) (98 mmol crude from previous step), Et₃N (40 mL) and 50% aq hydroxylamine (16 mL) in THF (250 mL) heated to 90° C. in a sealed pressure vessel for 1.5 h. After cooling to RT, the reaction was poured into water and extracted with EtOAC. The combined organic extracts were washed with brine and dried (Na2SO4). The solvent was removed in vacuo. The crude bis-oxime was dissolved in dioxane (200 mL) and Et₃N (35 mL) and heated to 150° C. in a sealed pressure vessel for 1.5 h. After allowing the reaction to cool to RT, the solvent was removed in vacuo to give a solid. Recrystallization from CH₂Cl₂ afforded the desired compound (17.3 g). MS (ES+) m/z 343(M+H)⁺.

### h) 1,1-Dimethylethyl [4-(7-bromo-4-chloro-1-ethyl-1H-imidazo[4,5-c]pyridin-2-yl)-1,2,5-oxadiazol-3-yl]carbamate

A solution of the compound of Example 10(g) (8.50 g, 24.7 mmol), pyridine (70 mL), di-t-butyl dicarbonate (21.5 g, 98.8 mmol) and DMAP (3.01 g, 24.7 mmol) in 1,2-dichloroethane (140 mL) was stirred at 85° C. in a sealed flask for 1 h. The product mixture was partitioned between EtOAc and 1N HCl. The layers were separated and the organic extract washed with 1N HCl, brine and dried (Na₂SO₄). The solvent was removed in vacuo and the resulting solid triturated with EtOAc to afford the desired compound as beige solid (5.06 g). The mother liquor was evaporated to dryness and treated with 2% trifluoroacetic acid in CH₂Cl₂ (100 mL) for 20 h. The reaction mixture was neutralized with sat NaHCO₃, washed with brine and dried (Na₂SO₄). The solvent was removed in vacuo and the residue was subjected to flash chromatography (20% EtOAc/hexane, silica gel) to afford an additional crop of the desired compound (2.45 g). The combined yield of the desired compound was 8.55 g (78%). MS (ES+) m/z 443(M+H)⁺.

### i) 1,1-Dimethylethyl [4-(4-chloro-1-ethyl-7-hydroxy-1H-imidazo[4,5-c]pyridin-2-yl)-1,2,5-oxadiazol-3-yl]carbamate

To a solution of the compound of Example 10(h) (2.00 g, 4.51 mmol) in THF (60 mL), at −100° C. was added n-BuLi (4.50 mL, 2.5 M in hexane, 11.3 mmol) dropwise. After five minutes, a solution of B(OMe)₃ (1.50 mL, 13.5 mmol) in THF (2 mL) was added. After 10 min., the cooling bath was removed. After 1.5 h, 3M NaOH (3 mL) and 30% w/w H₂O₂ (9 mL) were added to the reaction. After an additional 1 h, the reaction was quenched by adding EtOAc and washing sequentially with 1N HCl, H₂O and brine and then drying over Na₂SO₄. The solvent was removed in vacuo and the residue triturated with EtOAc to afford the desired compound (1.45 g). MS (ES+) m/z 381 (M+H)⁺.

### j) 1,1-Dimethylethyl [4-(1-ethyl-7-hydroxy-4-phenyl-1H-imidazo[4,5-c]pyridin-2-yl)-1,2,5-oxadiazol-3-yl]carbamate

The compound of Example 10(i) (1.40 g, 3.67 mmol), phenylboronic acid (0.90 g, 7.34 mmol) and Pd(PPh₃)₄ (0.24 g) were added to 1,4-dioxane (40 mL) and 2M Na₂CO₃ (4.04 mL, 8.1 mmol). The reaction vessel was purged with nitrogen, sealed and heated to 90° C. for 18 h. After allowing the reaction to cool to RT, the solids were removed by filtration. The filtrate was concentrated in vacuo and the residue subjected to flash chromatography (75% EtOAc/hexanes, silica gel) to give the desired compound (1.16 g). MS (ES+) m/z 423(M+H)⁺.

### k) 4-{1-Ethyl-4-phenyl-7-[(4-piperidinylmethyl)oxy]-1H-imidazo[4,5-c]pyridin-2-yl}-1,2,5-oxadiazol-3-amine

To a suspension of polymer-bound PPh₃ (0.96 g, 1.2 mmol/g loading, 1.15 mmol) in CH₂Cl₂ (10 mL), was added 1,1-dimethylethyl 4-(hydroxymethyl)-1-piperidinecarboxylate (0.50 g, 2.30 mmol) and DEAD (0.18 mL, 1.15 mmol) dropwise. After 30 min, the suspension was cooled to 0° C. A solution of the compound of Example 10(j) (0.10 g, 0.23 mmol) in CH₂Cl₂ (5 mL) was added. After 1 h at 0° C., solids were removed by filtration and exhaustively washed with CH₂Cl₂. The combined filtrates were concentrated in vacuo and the resulting residue subjected to flash chromatography (35% EtOAc/hexane, silica gel) to give the desired title compound as a di-t-butylcarbamate. MS (ES+) m/z 620(M+H)⁺.

The di-t-butyl carbamate obtained above was dissolved in TFA (2 mL) and CH₂Cl₂ (2 mL). After 2 h, the solvent was removed in vacuo and the residue subjected to preparative reverse phase HPLC (CH₃CN/water gradient, 0.1% TFA) to give 34 mg of the title compound as a white solid. MS (ES+) m/z 420(M+H)⁺.

### Example 11

### Preparation of 4-{(4-(3-chlorophenyl)-1-ethyl-7-[(4-piperidinylmethyl)oxy]-1H-imidazo-[4,5-c]pyridin-2-yl}-1,2,5-oxadiazol-3-amine trifluoroacetate

The title compound was prepared in an analogous manner to Example 10 by substituting 3-chlorophenylboronic acid for phenylboronic acid in step (j). MS(ES+) m/z 454.0 [M+H]⁺

### Example 12

### Preparation of 4-[7-[(4-aminobutyl)oxy]-4-(3-chlorophenyl)-1-ethyl-1H-imidazo-[4,5-c]pyridin-2-yl]-1,2,5-oxadiazol-3-amine trifluoroacetate

The title compound was prepared in an analogous manner to Example 10 by substituting 3-chlorophenylboronic acid for phenylboronic acid in step (j) and 1,1-dimethylethyl (4-hydroxybutyl)carbamate for 1,1-dimethylethyl 4-(hydroxymethyl)-1-piperidinecarboxylate in step (k). MS(ES+) m/z 428.0 [M+H]⁺

### Example 13

### Preparation of 4-(7-[(3-aminopropyl)oxy]-1-ethyl-4-phenyl-1H-imidazo[4,5-c]pyridin-2-yl)-1,2,5-oxadiazol-3-amine trifluoroacetate

The title compound was prepared in an analogous manner to Example 10 by substituting 1,1-dimethylethyl (4-hydroxypropyl)carbamate for 1,1-dimethylethyl 4-(hydroxymethyl)-1-piperidinecarboxylate in step (k). MS(ES+) m/z 380.0 [M+H]⁺

### Example 14

### Preparation of 4-[1-Ethyl-7-(piperidin-4-ylmethoxy)-1H-imidazo[4,5-c]pyridin-2-yl]-furazan-3-ylamine

### a) Ethyl-(3-nitropyridin-4-yl)amine

4-Methoxy-3-nitropyridine hydrochloride (11.2 g, 58.9 mmol) in ethanol (75 ml) was treated with a 70% solution of ethylamine in water (32 ml) and heated under reflux for 1 hour. Further ethylamine solution (32 ml) was added and the mixture heated under reflux for a further 2 hours. After cooling to room temperature, the solvent was removed in vacuo and the residue dissolved in ethyl acetate, washed (×3) with water and saturated aqueous sodium chloride solution, dried over sodium sulphate and concentrated in vacuo to afford the title compound (8.7 g, 88%); MS (ES+) m/e 168 [M+H]⁺.

### b) (3-Bromo-5-nitropyridin-4-yl)ethylamine

To a solution of the product of 14(a) (3.0 g, 17.9 mmol) in acetic acid (40 ml) was added bromine (3.12 g, 1 ml, 19.7 mmol) and the mixture was heated at 100° C. for 20 hours. After cooling the solvent was removed in vacuo and the residue was partitioned between dichloromethane and saturated sodium bicarbonate solution. The organic phase was washed with water (×3), dried and evaporated in vacuo. Purification of the residue by silica gel chromatography eluting with 50% dichloromethane in ethyl acetate afforded the title compound (1.9 g, 43%). ¹H NMR (DMSO-d₆) 8.73 (1H, s), 8.52 (1H, s), 7.0 (1H, br), 3.25 (2H, m), 1.16 (3H, t, J=7.2 Hz).

### c) 5-Bromo-N⁴-Ethylpyridine-3,4-diamine

A solution of the product of 14(b) 1 (0.5 g, 2 mmol) in ethanol (8 ml)/water (10 ml) was stirred at 60° C. and sodium dithionite (2.12 g, 12.2 mmol) was added portionwise. After 10 minutes the mixture was cooled to room temperature, and diluted with water and dichloromethane. The organic phase was dried and evaporated in vacuo, the residue was used directly in the next reaction; ¹H NMR (DMSO-d₆) 7.76 (1H, s), 7.75 (1H, s), 5.0 (2H, br), 4.46 (1H, t, J=9.6 Hz), 3.26 (2H, m), 1.06 (3H, t, J=7.2 Hz).

### d) 4-(7-Bromo-1-ethyl-1H-imidazo[4,5-c]pyridin-2-yl)furazan-3-ylamine

The product from of 14(c) (500 mg, 3.6 mmole) and ethyl cyanoacetate (620 mg, 5.5 mmol) were heated together at 190° C. for 20 minutes. After cooling to room temperature, the residue was purified by column chromatography eluting with 10% methanol in ethyl acetate.

The resultant product (200 mg, 1.1 mmol) in methanol (4 ml) and 2N hydrochloric acid (4 ml) was treated portionwise with sodium nitrite (150 mg, 2.2 mmol) and stirred at room temperature for 2 hours. The pH of the mixture was adjusted to 12 by addition of 50% sodium hydroxide solution and a 50% solution of hydroxylamine in water (3 ml) was added. The mixture was heated at 90° C. for 2.5 hours and the reaction allowed to cool to room temperature. The resulting precipitate was filtered and dried in vacuo to give the title compound of this step 14(d). MH (ES+) m/e 309/311 [M+H]⁺.

### e) 2-(4-Amino-furazan-3-yl)-1-ethyl-1H-imidazo[4,5c]pyridin-7-ol

A solution of the product of 14(d) (2.6 g, 8.41 mmol) in tetrahydrofuran (180 ml) at −78° C. was treated with a 2.5M solution of n-butyllithium (8.41 ml, 21.03 mmol) in hexanes. After the addition was complete the mixture was treated with trimethylborate (2.62 g, 25.23 mmol) and allowed to reach room temperature. After 1.5 hours at room temperature the reaction was carefully quenched with 3M aq. NaOH (12.5 ml) followed by a 30% aqueous hydrogen peroxide solution (4.3 ml). After 45 minutes the reaction was acidified with 2M hydrochloric acid and then applied to a SCX ion exchange column and eluted with methanol and then a mixture of methanol/0.880 ammonia (9:1). The basic fractions were then reduced and the solid residue was triturated with dichloromethane and filtered to afford the title compound of this step 14(e), (1.2 g, 58%); MS (ES+) m/e 247 [M+H]⁺.

### f) 4-[2-(4-Amino-furazan-3-yl)-1-ethyl-1H-imidazo[4,5-c]pyridin-7-yloxymethyl]-piperidine-1-carboxylic acid tert-butyl ester

A mixture of the product from 14(e) (0.1 g, 0.406 mmol) and K₂CO₃ (0.112 g, 0.812 mmol) in acetone (3 ml) at −78° C. was treated with 4-iodomethylpiperidine-1-carboxylic acid tert-butyl ester (Villaiobos, A; et al, J. Med. Chem., 1994, 37(17), 2721) (0.145 g, 0.447 mmol) and heated at reflux for 18 hours. A further portion of 4-iodomethylpiperidine-1-carboxylic acid tert-butyl ester (0.145 g, 0.447 mmol) was then added and the heating continued for a further 6 hours. The reaction was then cooled, poured into water, extracted with dichloromethane, dried with NaSO₄ and reduced. The residue was chromatographed on silica gel eluting with ethyl acetate to afford the title compound, (0.071 g, 39%); MS (ES+) m/e 444 [M+H]⁺.

### g) 4-[1-Ethyl-7-(piperidin-4-ylmethoxy)-1H-imidazo[4,5-c]pyridin-2-yl]-furazan-3-ylamine

The product from 14(f) (0.071 g, 0.16 mmol) was stirred in trifluoroacetic acid (0.5 ml) and dichloromethane (1 ml) at room temperature for 1 hour and the solution was then co-evaporated three times with dichloromethane. The residue was purified by silica gel chromatography eluting with 0.880 ammonia:methanol:dichloromethane (1:9:90), to afford the title compound, (0.046 g, 83%); MS (ES+) m/e 334 [M+H]⁺.

### Example 15—Methods

GW572016 is N-{3-Chloro-4-[(3-fluorobenzyl)oxy]phenyl}-6-[5-({[2-(methane sulphonyl)ethyl]amino}methyl)-2-furyl]-4-quinazolinamine ditosylate monohydrate.

GW589522 is (4-(3-Fluoro-benzyloxy)-3-bromophenyl)-(6-(5-((2-methanesulphonyl-ethylamino)-methyl)-furan-2-yl)quinazolin-4-yl)-amine.

GW583340 is (4-(3-Fluoro-benzyloxy)-3-chlorophenyl)-(6-(2-((2-methanesulphonyl-ethylamino)-methyl)-thiazol-4-yl)quinazolin-4-yl)-amine.

LY294002 is 2-(4-morpholinyl)-8-phenyl-4H-1-benzopyran-4-one and was obtained from Biomol Research Laboratories.

Wortmannin is fungal metabolite from Penicillium fumiculosum, which was obtained from Biomol Research Laboratories.

Compound of Example 8 is 2-(4-amino-1,2,5-oxadiazol-3-yl)-4-(3-chloro phenyl)-1-(cyclopropylmethyl)-N{2-[(phenylmethyl)amino]ethyl}-1H-imidazo[4,5-c]pyridine-7-carboxamide, trifluoroacetate salt.

Compound of Example 9 is 4-[1-Ethyl-7-(piperidin-4-yloxy)-1H-imidazo[4,5-c]pyridin-2-yl]-furazan-3-ylamine.

Compound Of Example 10 is 4-{1-ethyl-4-phenyl-7-[(3-piperidinylmethyl)oxy]-1H-imidazo[4,5-c]pyridin-2-yl}1,2,5-oxadiazol-3-amine trifluoroacetate.

Compound of Example 11 is 4-{4-(3-chlorophenyl)-1-ethyl-7-[(4-piperidinylmethyl)oxy]-1H-imidazo-[4,5-c]pyridin-2-yl}-1,2,5-oxadiazol-3-amine trifluoroacetate.

Compound of Example 12 is 4-[7-[(4-aminobutyl)oxy]-4-(3-chlorophenyl)-1-ethyl-1H-imidazo-[4,5-c]pyridin-2-yl]-1,2,5-oxadiazol-3-amine trifluoroacetate.

Compound of Example 13 is 4-{7-[(3-aminopropyl)oxy]-1-ethyl-4-phenyl-1H-imidazo[4,5-c]pyridin-2-yl}-1,2,5-oxadiazol-3-amine trifluoroacetate.

Compound of Example 14 is 4-[1-Ethyl-7-(piperidin-4-ylmethoxy)-1H-imidazo[4,5-c]pyridin-2-yl]-furazan-3-ylamine.

HN5 cells are LICR-LON-HN5 head and neck carcinoma cells, which were a gift from the Institute of Cancer Research, Surrey, U.K.

T47D cells are human breast ductal carcinoma cells originally obtained from the American Type Culture Collection.

MDA-MB468 cells are human breast adenocarcinoma cells originally obtained from the American Type Culture Collection.

Cell lines were grown in RPMI-1640 supplemented with 25 mM HEPES, 10 mM glutamine and 10% fetal bovine serum and maintained at 37° C. and 5% CO₂ in a humid incubator. Assays were performed in 96 well microtiter plates with optimum seeding densities for each cell line.

Apoptosis was measured using the Roche Cell Death ELISA^{Plus} kit (catalog 1 774 425) which detects fragmented nucleosomal DNA that is generated during apoptosis. A second assay was used to demonstrate caspase activation (Promega Apo-ONE™ Homogeneous Caspase-3/7 Assay, catalog G7791) which is an early event in the apoptotic cascade.

Synergistic interaction between compounds was analyzed by the median effect method described by Chou and Talalay (Adv. Enzyme Regul. 22: 27-55, 1984). Briefly, if the two compounds fit the mutually exclusive model of Chou, one calculates the combination index (CI) using the formula
CI=((D)₁/(Dₓ)₁)+((D)₂/(Dₓ)₂) (1)
where (D)₁ is the concentration of drug 1 in the combination that gives “x” percent apoptosis, (D)₂ is the same for drug 2, and (Dₓ)₁ and (Dₓ)₂ are the concentrations of drug 1 or 2 that give “x” percent apoptosis when used alone. (D)₁ and (D)₂ are known from the composition of the combination and (Dₓ)₁ and (Dₓ)₂ can be calculated from the equation
Dₓ=Dₘ*[fₐ/(1−fₐ)]^{1/m} (2)
where Dₘ is the concentration of drug giving 50% effect, fₐ is the fraction affected, and m is the slope from the median effect plot of log (fₐ/fᵤ) where fᵤ is the fraction unaffected versus log (D). A CI less than 1 indicates synergy, equal to 1 indicates additivity and greater than 1 antagonism.

Sensitization is measured as the ratio between observed and expected apoptosis or caspase activation from a combination of AKT kinase inhibitor and EGFR/erb inhibitor. The expected level of activity (Aₑ) is calculated by
Aₑ=1−((1−A₁)*(1−A₂)) (3)
where A₁ and A₂ are the activities of drugs 1 and 2 alone at the concentration used in the combination (Harvey, R. J., J. Theor. Biol. 74: 411-437, 1978). A sensitization ratio (SR) of 1.0 suggests that the two inhibitors are acting independently, and a value above 1.0 indicates sensitization.

### Example 16

Dosing with GW572016 and the PI3 Kinase Inhibitor LY294002

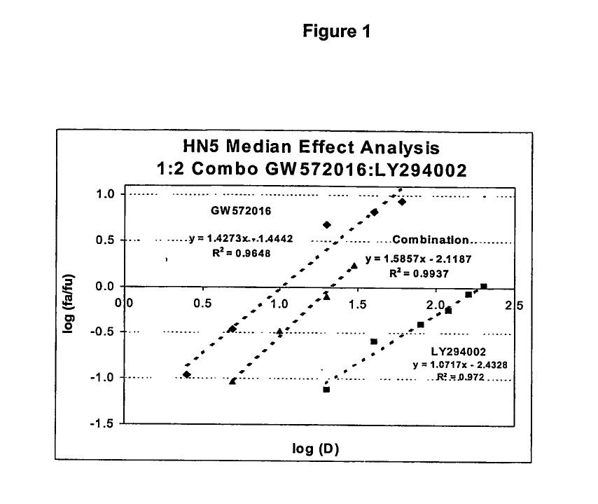

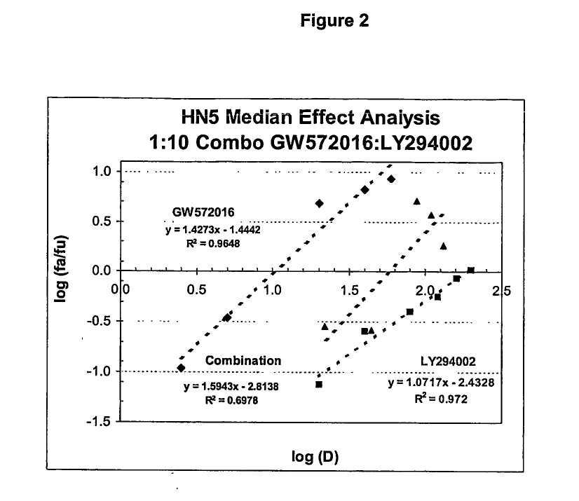

GW572016 and LY294002 alone and in 1:2 or 1:10 molar ratios (GW572016 to LY294002) were coincubated with HN5 cells for 24 h. Cell death was measured using the Roche Cell Death ELISA^{Plus} kit, and the median effect analysis was performed. The median effect plots are shown in FIG. 1 for the 1:2 combination and in FIG. 2 for the 1:10 combination. Calculations of Dₘ and CI are presented in Table 1 for the 1:2 and 1:10 combinations; the CI values of 0.78 and 0.80 for the two combinations indicated synergism in inducing apoptosis.

TABLE 1
Combination indices for 1:2 and 1:10 combinations
of GW572016 and LY294002 added to HN5 cells.

| | | | | |
|---|---|---|---|---|
| System | M | B | Dₘ, μM | Cl |

1:2 Combination

| | | | | |
|---|---|---|---|---|
| GW572016 alone | 1.427 | −1.444 | 10.3 | |
| LY294002 alone | 1.072 | −2.433 | 186 | |
| Combo 1:2 | 1.586 | −2.119 | 21.7 | 0.781 |
| GW572016 in combo | | | 7.2 | |
| LY294002 in combo | | | 14.5 | |

1:10 Combination

| | | | | |
|---|---|---|---|---|
| GW572016 alone | 1.427 | −1.444 | 10.3 | |
| LY294002 alone | 1.072 | −2.433 | 186 | |
| Combo 1:10 | 1.594 | −2.814 | 58.2 | 0.799 |
| GW572016 in combo | | | 5.3 | |
| LY294002 in combo | | | 52.9 | |

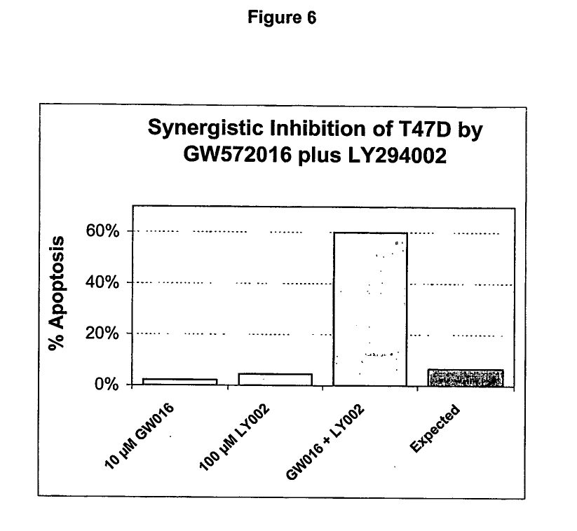

Synergism between GW572016 and LY294002 was also demonstrated using the sensitization ratio method. T47D cells were incubated with varied concentrations of GW572016 for 24 h followed by an additional 4 h incubation with varied concentrations of LY294002. Apoptosis was measured using the Roche Cell Death ELISA^{Plus} kit, and the relative percentage of apoptosis was determined for each combination. Sensitization ratios were calculated as described above and are listed in Table 2. When 10 μM GW572016 was combined with 20, 50 or 100 μM LY294002, SR values 3.2, 7.3 and 9.0, respectively, indicated significant synergism. At lower concentrations of GW572016, lesser degrees of synergism were observed. FIG. 6 graphically illustrates the significant apoptosis induced by a combination of 10 μM GW572016 and 100 μM LY294002 when the drugs separately had little effect. Similar results were seen with the MDA-MB468 cell line (data not shown).

TABLE 2
Sensitization ratios for combinations of
GW572016 and LY294002 in T47D cells.

| μM | | Net avg | Net | Expect | Sensitization |
|---|---|---|---|---|---|
| GW-016 | LY-002 | A₄₀₅ nm | Apoptosis | Apoptosis | Ratio |
| 0 | 0 | 0.007 | 0.0% | | |
| 2 | 0 | 0.013 | 0.4% | | |
| 5 | 0 | 0.013 | 0.3% | | |
| 10 | 0 | 0.042 | 2.2% | | |
| 0 | 20 | 0.041 | 2.2% | | |
| 2 | 20 | 0.040 | 2.1% | 2.5% | 0.8 |
| 5 | 20 | 0.071 | 4.1% | 2.5% | 1.6 |
| 10 | 20 | 0.222 | 13.8% | 4.3% | 3.2 |
| 0 | 50 | 0.063 | 3.6% | | |
| 2 | 50 | 0.088 | 5.2% | 4.0% | 1.3 |
| 5 | 50 | 0.117 | 7.1% | 3.9% | 1.8 |
| 10 | 50 | 0.659 | 42.1% | 5.8% | 7.3 |
| 0 | 100 | 0.077 | 4.5% | | |
| 2 | 100 | 0.114 | 6.9% | 4.9% | 1.4 |
| 5 | 100 | 0.244 | 15.3% | 4.8% | 3.2 |
| 10 | 100 | 0.935 | 59.9% | 6.6% | 9.0 |

### Example 17

Dosing with GW589522 and the PI3 Kinase Inhibitor LY294002

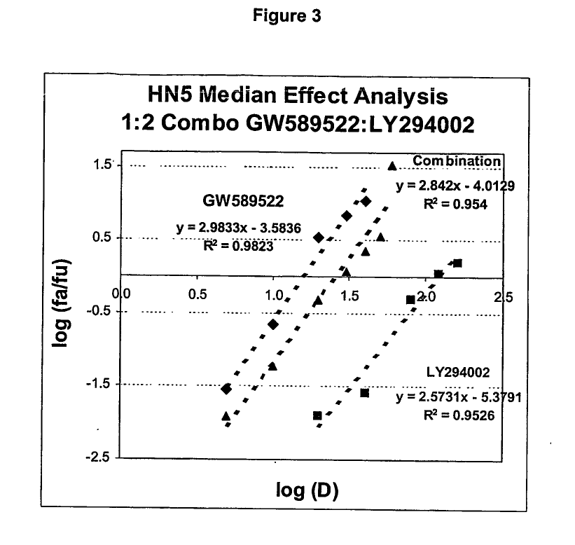

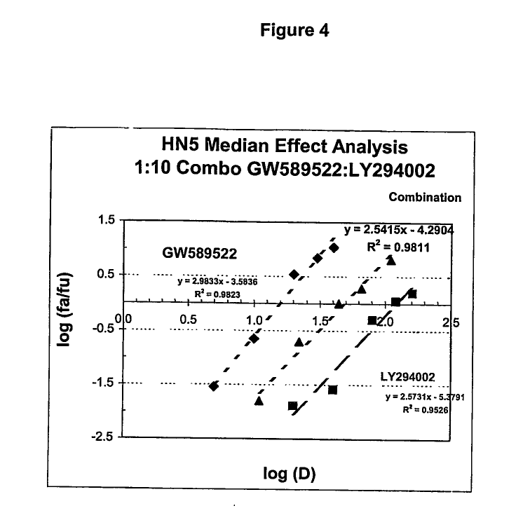

GW589522 and LY294002 alone and in 1:2 or 1:10 molar ratios (GW5789522 to LY294002) were coincubated with HN5 cells for 24 h. Cell death was measured using the Roche Cell Death ELISA^{Plus} kit, and median effect analysis was performed. The median effect plots are shown in FIG. 3 for the 1:2 combination and in FIG. 4 for the 1:10 combination. Calculations of Dₘ and CI are presented in Table 3 for the 1:2 and 1:10 combinations; the CI values of 0.68 and 0.64 for the two combinations indicated synergism in inducing apoptosis.

TABLE 3
Combination indices for 1:2 and 1:10 combinations
of GW589522 and LY294002 added to HN5 cells.

| | | | | |
|---|---|---|---|---|
| System | M | B | Dₘ, μM | Cl |

1:2 Combination

| | | | | |
|---|---|---|---|---|
| GW589522 alone | 2.983 | −3.584 | 15.9 | |
| LY294002 alone | 2.573 | −5.379 | 123.2 | |
| Combo 1:2 | 2.842 | −4.013 | 25.8 | 0.681 |
| GW589522 in combo | | | 8.6 | |
| LY294002 in combo | | | 17.2 | |

1:10 Combination

| | | | | |
|---|---|---|---|---|
| GW589522 alone | 2.983 | −3.584 | 15.9 | |
| LY294002 alone | 2.573 | −5.379 | 123.2 | |
| Combo 1:10 | 2.542 | −4.290 | 48.8 | 0.639 |
| GW589522 in combo | | | 4.4 | |
| LY294002 in combo | | | 44.3 | |

### Example 18

Dosing with GW583340 and the PI3 Kinase Inhibitor LY294002

Synergism between GW583340 and LY294002 was demonstrated using the sensitization ratio method. MDA-MB468 cells were incubated with varied concentrations of GW583340 for 24 h followed by an additional 4 h incubation with varied concentrations of LY294002. Apoptosis was measured using the Roche Cell Death ELISA^{Plus} kit, and the relative percentage of apoptosis was determined for each combination. Sensitization ratios were calculated as described above and are listed in Table 4. When 1, 2, or 5 μM GW583340 were combined with 100 μM LY294002, SR values 3.4, 4.2 and 5.0, respectively, indicated significant synergism. At lower concentrations of LY294002, lesser degrees of synergism were observed. Similar results were seen with the T47D cell line.

TABLE 4
Sensitization ratios for combinations of
GW583340 and LY294002 in MDA-MB468 cells.

| μM | | Net avg | Net | Expect | Sensitization |
|---|---|---|---|---|---|
| GW-340 | LY-002 | A₄₀₅ nm | Apoptosis | Apoptosis | Ratio |
| 0 | 0 | 0.237 | 0.0% | | |
| 0 | 4 | 0.294 | 4.8% | | |
| 0 | 10 | 0.314 | 6.5% | | |
| 0 | 20 | 0.338 | 8.6% | | |
| 0 | 50 | 0.344 | 9.0% | | |
| 0 | 100 | 0.399 | 13.7% | | |
| 1 | 0 | 0.294 | 4.8% | | |
| 1 | 4 | 0.301 | 5.4% | 9.4% | 0.6 |
| 1 | 10 | 0.393 | 13.2% | 11.1% | 1.2 |
| 1 | 20 | 0.383 | 12.3% | 13.0% | 0.9 |
| 1 | 50 | 0.489 | 21.3% | 13.4% | 1.6 |
| 1 | 100 | 0.950 | 60.1% | 17.8% | 3.4 |
| 2 | 0 | 0.286 | 4.2% | | |
| 2 | 4 | 0.374 | 11.6% | 8.8% | 1.3 |
| 2 | 10 | 0.438 | 17.0% | 10.4% | 1.6 |
| 2 | 20 | 0.461 | 18.9% | 12.4% | 1.5 |
| 2 | 50 | 0.654 | 35.2% | 12.8% | 2.7 |
| 2 | 100 | 1.096 | 72.4% | 17.3% | 4.2 |
| 5 | 0 | 0.325 | 7.4% | | |
| 5 | 4 | 0.454 | 18.3% | 11.9% | 1.5 |
| 5 | 10 | 0.463 | 19.1% | 13.5% | 1.4 |
| 5 | 20 | 0.659 | 35.6% | 15.4% | 2.3 |
| 5 | 50 | 1.021 | 66.1% | 15.8% | 4.2 |
| 5 | 100 | 1.423 | 100.0% | 20.1% | 5.0 |

### Example 19

Dosing with GW572016 and the AKT Inhibitor of Example 9.

Synergism between GW572016 and the compound of Example 9 was demonstrated using the sensitization ratio method. HN5 cells were incubated with varied concentrations of GW572016 for 24 h followed by an additional 4 h incubation with varied concentrations of the Example 9 compound. Apoptosis was measured using the Roche Cell Death ELISA^{Plus} kit, and the relative percentage of apoptosis was determined for each combination. Sensitization ratios were calculated as described above and are listed in Table 5. Significant synergism was observed with GW572016 concentration as low as 2 μM when combined with 8 μM of the compound of Example 9 (SR=11). The degree of synergism increased as the concentrations of the two compounds increased.

TABLE 5
Sensitization ratios for combinations of GW572016
and Compound of Example 9 in HN5 cells.

| | | | |
|---|---|---|---|
| μM | Net | Expect | Sensitization |

| GW-016 | Ex 9 | Apoptosis | Apoptosis | Ratio |
|---|---|---|---|---|
| 0 | 0 | 0.0% | | |
| 0 | 2 | 2.7% | | |
| 0 | 4 | 1.8% | | |
| 0 | 8 | 3.9% | | |
| 0 | 16 | 3.7% | | |
| 0 | 20 | 4.7% | | |
| 2 | 0 | −1.0% | | |
| 2 | 2 | 2.1% | 1.7% | 1.2 |
| 2 | 4 | 2.9% | 0.9% | 3.4 |
| 2 | 8 | 33.2% | 3.0% | 11.1 |
| 2 | 16 | 58.9% | 2.8% | 21.2 |
| 2 | 20 | 63.0% | 3.8% | 16.7 |
| 4 | 0 | 2.5% | | |
| 4 | 2 | 3.4% | 5.1% | 0.7 |
| 4 | 4 | 8.3% | 4.3% | 2.0 |
| 4 | 8 | 65.6% | 6.3% | 10.4 |
| 4 | 16 | 86.1% | 6.1% | 14.1 |
| 4 | 20 | 100% | 7.1% | 14.2 |
| 10 | 0 | 5.1% | | |
| 10 | 2 | 14.6% | 7.7% | 1.9 |
| 10 | 4 | 31.5% | 6.8% | 4.6 |
| 10 | 8 | 86.6% | 8.8% | 9.8 |
| 10 | 16 | 91.4% | 8.6% | 10.6 |
| 10 | 20 | 97.8% | 9.6% | 10.2 |

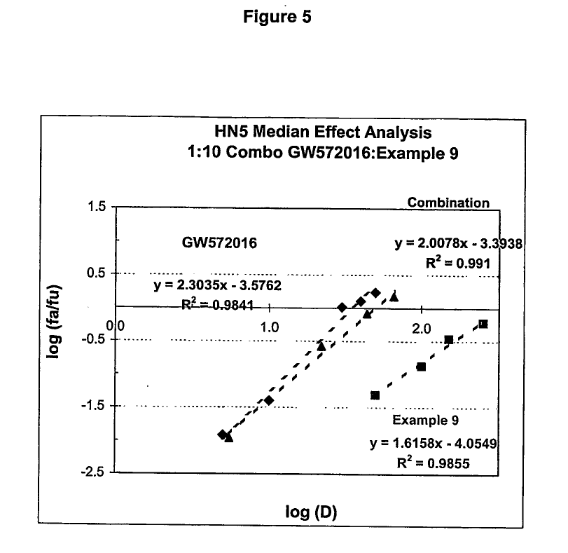

Synergism between GW572016 and the Example 9 Akt inhibitor was also demonstrated using median effect analysis. HN5 cells were incubated in the absence or presence of varied concentrations of GW572016 for 24 h followed by an an additional 4 h incubation with the Example 9 compound alone or in 1:2, 1:10 and 1:20 molar ratios (GW572016 to Example 9 compound). Cell death was measured using the Roche Cell Death ELISA^{Plus} kit. The median effect plots are shown in FIG. 5 for the 1:10 combination. The CI values of 0.29, 0.26 and 0.29 for the respective combinations indicated synergism in inducing apoptosis. Similar results were obtained when the caspase activation was used in place of the cell death assay with CI values of 0.42, 0.37 and 0.39 for the 1:2,1:10 and 1:20 combinations, respectively.

### Example 20

Dosing with GW589522 and the PI3 Kinase Inhibitor Wortmannin.

Synergism between GW589522 and Wortmannin was demonstrated using the sensitization ratio method. HN5 cells were incubated in the absence or with 5 or 10 μM GW589522 for 24 h followed by an additional 4 h incubation with 40 μM Wortmannin. Apoptosis was measured using the Roche Cell Death ELISA^{Plus} kit, and the relative percentage of apoptosis was determined for each combination. Sensitization ratios were calculated as described above. In the presence of 5 μM GW589522, the SR averaged from four plates was 7.9±0.9; in the presence of 10 μM GW589522, the average SR was 5.8±0.4. Similar results were seen with T47D cells where the average SR were 3.4±0.7 with 5 μM and 11.7±3.8 with 10 μM GW589522.

### Example 21

Dosing with GW589522 and the AKT Inhibitor of Example 9.

Synergism between GW589522 and the compound of Example 9 was demonstrated using the sensitization ratio method. HN5 cells were incubated with varied concentrations of GW589522 for 24 h followed by an additional 4 h incubation with varied concentrations of the Example 9 compound. Apoptosis was measured using the Roche Cell Death ELISA^{Plus} kit, and the relative percentage of apoptosis was determined for each combination. Sensitization ratios were calculated as described above and are listed in Table 6. Significant synergism was observed with the GW589522 concentration as low as 1.6 μM when combined with 8 μM of the Example 9 compound (SR=3.7).

TABLE 6
Sensitization ratios for combinations of GW589522
and the Akt inhibitor of Example 9 in HN5 cells.

| | | | |
|---|---|---|---|
| μM | Net | Expect | Sensitization |

| GW-522 | Ex 9 | Apoptosis | Apoptosis | Ratio |
|---|---|---|---|---|
| 0 | 0 | 8.0% | | |
| 0 | 0.5 | 6.3% | | |
| 0 | 2 | 8.7% | | |
| 0 | 8 | 17.9% | | |
| 1.56 | 0 | −1.0% | | |
| 1.56 | 0.5 | 2.1% | 5.3% | 0.4 |
| 1.56 | 2 | 2.9% | 7.8% | 0.4 |
| 1.56 | 8 | 63.0% | 17.0% | 3.7 |
| 3.12 | 0 | 2.5% | | |
| 3.12 | 0.5 | 3.4% | 8.6% | 0.4 |
| 3.12 | 2 | 8.3% | 10.9% | 0.8 |
| 3.12 | 8 | 100% | 19.9% | 5.0 |
| 6.25 | 0 | 2.5% | | |
| 6.25 | 0.5 | 3.4% | 8.6% | 0.4 |
| 6.25 | 2 | 8.3% | 10.9% | 0.8 |
| 6.25 | 8 | 100% | 19.9% | 5.0 |
| 12.5 | 0 | 5.1% | | |
| 12.5 | 0.5 | 14.6% | 11.0% | 1.3 |
| 12.5 | 2 | 31.5% | 13.3% | 2.4 |
| 12.5 | 8 | 97.8% | 22.0% | 4.4 |

### Example 22

GW589522 and Various AKT Inhibitors are Synergistic.

Synergy between GW589522 and several inhibitors of AKT kinase was shown using the sensitization ratio method. After 24 h exposure of HN5 to 5 μM GW589522, cells were exposed for 4 h to various concentrations of several compounds that had demonstrated inhibitory activity towards AKT kinase as shown in Table 7. Apoptosis was measured using the Roche Cell Death ELISA^{Plus} kit, the relative percentage of apoptosis was determined for each combination and the sensitization ratio calculated as described above. The SR values listed in Table 8 indicate that GW589522 synergized with each compound in inducing apoptosis in HN5. In the T47D cell line that is less sensitive in demonstrating synergy, the compound of Example 8 gave an SR of 7.1 at 25 μM.

TABLE 7
Inhibition of AKT isoforms by various compounds.

| | Enzyme IC₅₀, μM | | |
|---|---|---|---|
| Compound | AKT1 | AKT2 | AKT3 |
| Ex 9 (tri-HCl salt) | 0.05 | 0.27 | 0.19 |
| Ex 9 | 0.13 | 0.39 | 0.41 |
| Ex 8 | 8.32 | 7.94 | 0.45 |
| Ex 10 | 0.01 | 0.03 | 0.01 |
| Ex 11 | 0.03 | 0.02 | 0.01 |
| Ex 12 | 0.04 | 0.04 | 0.01 |
| Ex 13 | 0.04 | 0.02 | 0.01 |
| Ex 14 | 0.07 | 0.27 | 0.51 |

TABLE 8
Sensitization ratios of AKT inhibitors
exhibiting synergy with GW589522.
Sensitization Ratio in HN5

| Compound | with 5 μM GW589522A | |
|---|---|---|
| Ex 9 (tri-HCl salt) | 10 @ 8 | μM |
| Ex 9 | 2.6 @ 4 | μM |
| Ex 8 | 27 @ 25 | μM |
| Ex 10 | 3.7 @ 10 | μM |
| Ex 11 | 20 @ 2.5 | μM |
| Ex 11 | 25 @ 5 | μM |
| Ex 12 | 3.6 @ 25 | μM |
| Ex 13 | 12 @ 5 | μM |
| Ex 14 | 4.5 @ | μM |

Values are SR @ concentration tested

What is claimed is:

1-2. (canceled)

3. A method of testing a susceptible cancer in a mammal, comprising: administering to said mammal therapeutically effective amounts of (i) a compound of formula (II): 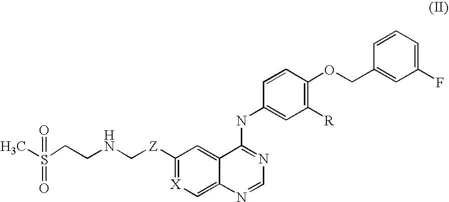
or salt or solvates thereof, wherein R is —Cl or —Br, X is CH, N, or CF, and Z is thiazole or furan; and
(ii) at least one PI3K and an Akt inhibitor.

4. A method of testing a susceptible cancer in a mammal, comprising: administering to said mammal therapeutically effective amounts of (i) a compound of formula (III): 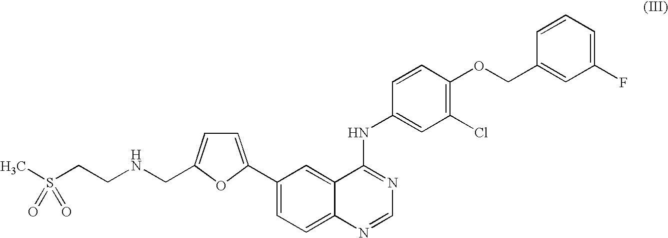
or salts or solvates thereof; and
(ii) at least one of a PI3K and an Akt inhibitor.

5-6. (canceled)

7. A cancer treatment combination, comprising: therapeutically effective amounts of (i) a compound of formula (II): 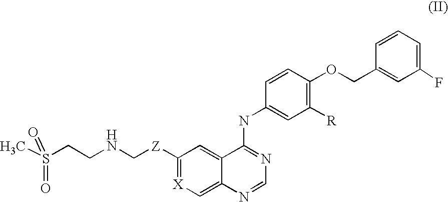
or salt or solvates thereof wherein R is —Cl or —Br, X is CH, N, or CF, and Z is thiazole or furan; and
(ii) at least one PI3K and an Akt inhibitor.

8. A cancer treatment combination, comprising: therapeutically effective amounts of (i) a compound of formula (III): 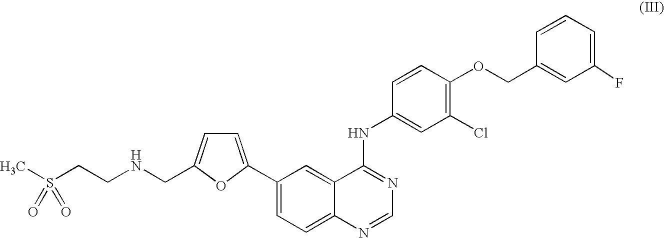
or salts or solvates thereof; and
(ii) at least one PI3K and an Akt inhibitor.

9-10. (canceled)

11. A method as claimed in claim 4, wherein the compound is a ditosylate salt of the compound of formula (III).

12. A method as claimed in claim 4, wherein the compound is a monohydrate ditosylate salt of the compound of formula (III).

13. A method as claimed in claim 4, wherein the compound is an anhydrous ditosylate salt of the compound of formula (III).

14. A combination as claimed in claim 8, wherein the compound is a ditosylate salt of the compound of formula (III).

15. A combination as claimed in claim 8, wherein the compound is a monohydrate ditosylate salt of the compound of formula (III).

16. A combination as claimed in claim 8, wherein the compound is an anhydrous ditosylate salt of the compound of formula (III).

17. A method as claimed in claim 4, wherein the at least one PI3K and an Akt inhibitor is a PI3K inhibitor.

18. A method as claimed in claim 4, wherein the at least one PI3K and an Akt inhibitor is an Akt inhibitor.

19. A method as claimed in claim 8, wherein the at least one PI3K and an Akt inhibitor is a PI3K inhibitor.

20. A method as claimed in claim 8, wherein the at least one PI3K and an Akt inhibitor is an Akt inhibitor.

21. A method of treating a susceptible cancer in a mammal, comprising: administering to said mammal therapeutically effective amounts of (i) a monohydrated form of a ditosylate salt of a compound of formula (III): 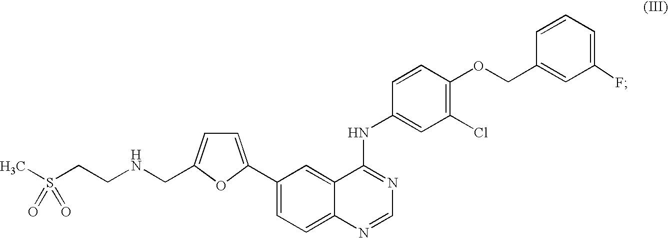
and
(ii) an Akt inhibitor.

22. A cancer treatment combination, comprising: administering to said mammal therapeutically effective amounts of (i) a monohydrated form of a ditosylated salt of a compound of formula (III): 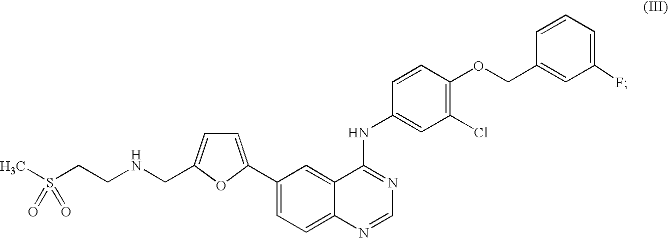
and
(ii) an Akt inhibitor.

23. A method of treating a susceptible cancer in a mammal, comprising: administering to said mammal therapeutically effective amounts of (i) a monohydrated form of a ditosylate salt of a compound of formula (III): 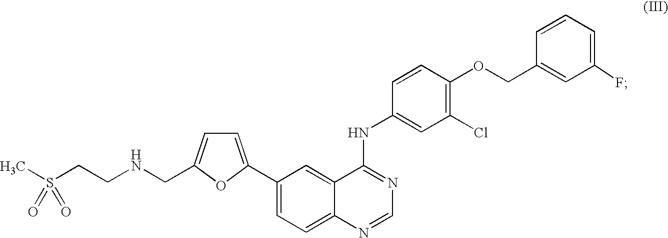
and
(ii) an Akt inhibitor.

24. A cancer treatment combination, comprising: administering to said mammal therapeutically effective amounts of (i) a monohydrated form of a ditosylated salt of a compound of formula (III): 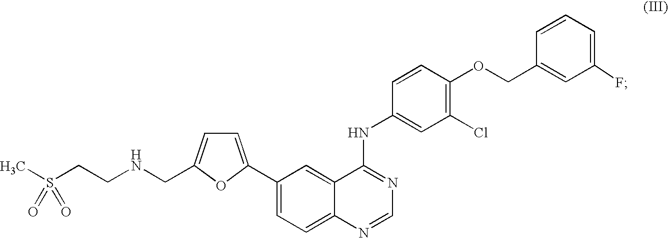
and
(ii) an Akt inhibitor.

25. A method of treating a susceptible cancer in a mammal, comprising: administering to said mammal therapeutically effective amounts of (i) a monohydrated form of a ditosylate salt of a compound of formula (III): 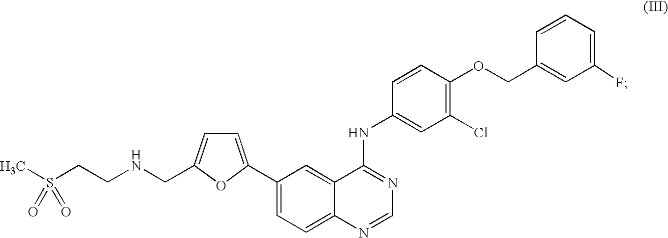
and
(ii) an Akt inhibitor.

26. A cancer treatment combination, comprising: administering to said mammal therapeutically effective amounts of (i) a monohydrated form of a ditosylated salt of a compound of formula (III): 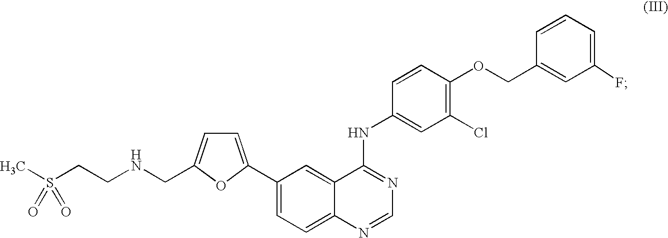
and
(ii) an Akt inhibitor.
